(19) United States
(12) Patent Application Publication
(10) Pub. No.: US 2026/0226116 A1
(43) Pub. Date: Aug. 6, 2026

(54) TREATMENT OF FRAGILE X SYNDROME

(71) Applicant: UTI LIMITED PARTNERSHIP, Calgary (CA)

(72) Inventors: Raymond W. TURNER, Calgary (CA); Xiaogin ZHAN, Calgary (CA); Hadhimulya ASMARA, Calgary (CA)

(21) Appl. No.: 19/146,332

(22) PCT Filed: Jan. 11, 2024
(86) PCT No.: PCT/CA2024/050031

§ 371 Date: Jul. 8, 2025

Related U.S. Application Data
(60) Provisional application No. 63/479,440, filed on Jan. 11, 2023.

(51) Int. Cl. C07K 14/47; A61K 31/155; A61K 31/165; A61K 38/00; A61K 45/06; A61P 25/00

(52) U.S. Cl. CPC C07K 14/4702; A61K 31/155; A61K 31/165; A61K 45/06; A61P 25/00; A61K 38/00; C07K 2319/10; C07K 2319/21; C07K 2319/41

(57) ABSTRACT

The present disclosure relates generally to the treatment of Fragile X Syndrome.

### FIELD

The present disclosure relates generally to the treatment of Fragile X Syndrome.

### BACKGROUND

Fragile X Syndrome is the most common genetic cause of intellectual disability and incidence of autism spectrum disorder. Fragile X results from an inordinate number of CGG repeats (>200) on the UTR region of the fmrp1 gene on the X chromosome that leads to hypermethylation and block of transcription of the fmrp1 gene. As a result, there is a loss of expression of Fragile X Messenger Ribonucleoprotein (FMRP) (previously referred to as Fragile X Mental Retardation Protein) that is known to regulate translation or activity of multiple proteins required to exhibit normal levels of synaptic plasticity. The fact that this disorder arises from the loss of a single protein provides an incentive to understand how it disrupts synaptic plasticity and to identify a treatment strategy that either restores FMRP or blocks secondary adverse events in order to reduce behavioural dysfunctions of Fragile X syndrome. Central to these tests is extensive use of a FMRP−/− mouse line that effectively recapitulates the key genetic disruption of FMRP expression, with many traits similar to that of Fragile X patients that harbor the full genetic mutation.

### SUMMARY

In one aspect there is provided a recombinant fusion polypeptide, comprising: a FMRP(444-632) polypeptide and a cell penetrating polypeptide, or fragment or variant thereof.

In one example, said cell penetrating polypeptide comprises a tat polypeptide.

In one example, further comprising a linker positioned between said cell penetrating polypeptide and said FMRP(444-632) polypeptide.

In one example, wherein the linker is GSGGG (SEQ ID NO: 11).

In one example, where said tat polypeptide comprises YGRKKRRQRRR (SEQ ID NO: 4).

In one example, further comprising a HIS polypeptide.

In one example, wherein said HIS polypeptide comprises HHHHHH (SEQ ID NO: 12) or MGGSHHHHHHGMAS (SEQ ID NO: 5).

In one example, further comprising an immunogenic tag.

In one example, wherein the immunogenic tag is a c-myc tag.

In one example, wherein the c-myc tag is EQKLISEEDL (SEQ ID NO: 6).

In one example, wherein the FMRP(444-632) polypeptide is

(SEQ ID NO: 7)
MGASSRPPPNRTDKEKSYVTDDGQGMGRGSRPYRNRGHGRRGPGYTSGTNSEASNA
SETESDHRDELSDWSLAPTEEERESFLRRGDGRRRGGGGRGQGGRGRGGGFKGND
DHSRTDNRPRNPREAKGRTTDGSLQIRVDCNNERSVHTKTLQNTSSEGSRLRTGKDRN
QKKEKPDSVDGQQPLVNGVP.

In one example, wherein the sequence is

(SEQ ID NO: 3)
MGGSHHHHHHGMASEQKLISEEDLMGASSRPPPNRTDKEKSYVTDDGQGMGRGSRP
YRNRGHGRRGPGYTSGTNSEASNASETESDHRDELSDWSLAPTEEERESFLRRGDGR
RRGGGGRGQGGRGRGGGFKGNDDHSRTDNRPRNPREAKGRTTDGSLQNTSSEGSR
LRTGKDRNQKKEKPDSVDGQQPLVNGVPGSGGGYGRKKRRQRRR.

In one example, wherein said fusion polypeptide comprises a variant fusion polypeptide sequence that is at least 80% identical to said fusion polypeptide.

In one aspect there is provided a polynucleotide molecule comprising or consisting of a sequence that encodes a recombinant fusion polypeptide according to any one of claims 1 to 13.

In one aspect there is provided a vector comprising the polynucleotide molecule of claim 14.

In one aspect there is provided a cell comprising the polynucleotide molecule of claim 14.

In one aspect there is provided a cell comprising the vector of claim 15.

In one aspect there is provided a pharmaceutical composition comprising a recombinant fusion polypeptide of any one of claims 1-13, a polynucleotide molecule of claim 14, or a vector of claim 15, and a pharmaceutically acceptable carrier.

In one aspect there is provided a method of treatment of a subject having or suspected of having Fragile X Syndrome, comprising: administering a recombinant fusion polypeptide of any one of claims 1 to 13, a polynucleotide molecule of claim 14, a vector of claim 15, or a pharmaceutical composition of claim 18, to said subject.

In one example, further comprising administration of minocycline, metformin, and/or blockers of extracellular signal-regulated kinase (ERK).

In one example, wherein said subject is a human.

In one aspect there is provided a recombinant fusion polypeptide, comprising: a FMRP(399-570) polypeptide and a cell penetrating polypeptide, or fragment or variant thereof.

In one example, where said cell penetrating polypeptide comprises a tat polypeptide.

In one example, further comprising a linker positioned between said cell penetrating polypeptide and said FMRP(399-570) polypeptide.

In one example, wherein the linker is GSGGG (SEQ ID NO: 11).

In one example, where said tat polypeptide comprises YGRKKRRQRRR SEQ ID NO: 4).

In one example, further comprising a HIS polypeptide.

In one example, wherein said HIS polypeptide comprises HHHHHH (SEQ ID NO: 12) or MGGSHHHHHHGMAS (SEQ ID NO: 5).

In one example, further comprising an immunogenic tag.

In one example, wherein the immunogenic tag is a c-myc tag.

In one example, wherein the c-myc tag is EQKLISEEDL (SEQ ID NO: 6).

In one example, wherein the FMRP(399-570) polypeptide is

(SEQ ID NO 8)
MGASSRPPPNRTDKEKSYVTDDGQGMGRGSRPYRNRGHGRRGPGYTSGTNSEASNA
SETESDHRDELSDWSLAPTEEERESFLRRGDGRRRGGGGRGQGGRGRGGGFKGND
DHSRTDNRPRNPREAKGRTTDGSLQNTSSEGSRLRTGKDRNQKKEKPDSVDGQQPLV
NGVP.

In one example, wherein the sequence is

(SEQ ID NO: 3)
MGGSHHHHHHGMASEQKLISEEDLMGASSRPPPNRTDKEKSYVTDDGQGMGRGSRP
YRNRGHGRRGPGYTSGTNSEASNASETESDHRDELSDWSLAPTEEERESFLRRGDGR
RRGGGGRGQGGRGRGGGFKGNDDHSRTDNRPRNPREAKGRTTDGSLQNTSSEGSR
LRTGKDRNQKKEKPDSVDGQQPLVNGVPGSGGGYGRKKRRQRRR.

In one example, wherein said fusion polypeptide comprises a variant fusion polypeptide sequence that is at least 80% identical to said fusion polypeptide.

In one aspect there is provided a polynucleotide molecule comprising or consisting of a sequence that encodes a recombinant fusion polypeptide according to any one of claims 22 to 34.

In one aspect there is provided a vector comprising the polynucleotide molecule of claim 35.

In one aspect there is provided a cell comprising the polynucleotide molecule of claim 35.

In one aspect there is provided a cell comprising the vector of claim 36.

In one aspect there is provided a pharmaceutical composition comprising a recombinant fusion polypeptide of any one of claims 22-34, a polynucleotide molecule of claim 35, or a vector of claim 36, and a pharmaceutically acceptable carrier.

In one aspect there is provided a method of treatment of a subject having or suspected of having Fragile X Syndrome, comprising: administering a recombinant fusion polypeptide of any one of claims 22 to 34, a polynucleotide molecule of claim 35, a vector of claim 36, or a pharmaceutical composition of claim 39, to said subject.

In one example, further comprising administration of minocycline, metformin, and/or blockers of extracellular signal-regulated kinase (ERK).

In one example, wherein said subject is a human.

In one aspect there is provided a recombinant fusion polypeptide, comprising: a FMRP(205-399) polypeptide and a cell penetrating polypeptide, or fragment or variant thereof.

In one example, where said cell penetrating polypeptide comprises a tat polypeptide.

In one example, further comprising a linker positioned between said cell penetrating polypeptide and said FMRP(205-399) polypeptide.

In one example, wherein the linker is GSGGG (SEQ ID NO:11).

In one example, where said tat polypeptide comprises YGRKKRRQRRR (SEQ ID NO: 4).

In one example, further comprising a HIS polypeptide.

In one example, wherein said HIS polypeptide comprises HHHHHH (SEQ ID NO: 12) or MGGSHHHHHHGMAS (SEQ ID NO: 5).

In one example, further comprising an immunogenic tag.

In one example, wherein the immunogenic tag is a c-myc tag.

In one example, wherein the c-myc tag is EQKLISEEDL (SEQ ID NO: 6).

In one example, wherein the FMRP(205-399) polypeptide is

(SEQ ID NO: 1)
MSKQLESSRQLASRFHEQFIVREDLMGLAIGTHGANIQQARKVPGVTAIDLDEDTCTFHI
YGEDQDAVKKARSFLEFAEDVIQVPRNLVGKVIGKNGKLIQEIVDKSGVVRVRIEAENEK
NVPQEEVLVASSVVAGESQKPELKAWQGMVPFVFVGTKDSIANATVLLDYHLNYLKEVD
QLRLERLQIDEQLRQI.

In one example, wherein the sequence is

(SEQ ID NO: 9)
MGGSHHHHHHGMASEQKLISEEDLMSKQLESSRQLASRFHEQFIVREDLMGLAIGTHG
ANIQQARKVPGVTAIDLDEDTCTFHIYGEDQDAVKKARSFLEFAEDVIQVPRNLVGKVIGK
NGKLIQEIVDKSGVVRVRIEAENEKNVPQEEVLVASSVVAGESQKPELKAWQGMVPFVF
VGTKDSIANATVLLDYHLNYLKEVDQLRLERLQIDEQLRQIGSGGGYGRKKRRQRRR.

In one example, wherein said fusion polypeptide comprises a variant fusion polypeptide sequence that is at least 80% identical to said fusion polypeptide, or fragments or variants thereof.

In one aspect there is provided a polynucleotide molecule comprising or consisting of a sequence that encodes a recombinant fusion polypeptide according to any one of claims 43 to 55.

In one aspect there is provided a vector comprising the polynucleotide molecule of claim 56.

In one aspect there is provided a cell comprising the polynucleotide molecule of claim 56.

In one aspect there is provided a cell comprising the vector of claim 57.

In one aspect there is provided a pharmaceutical composition comprising a recombinant fusion polypeptide of any one of claims 43-55, a polynucleotide molecule of claim 56, or a vector of claim 57, and a pharmaceutically acceptable carrier.

In one aspect there is provided a method of treatment of a subject having or suspected of having Fragile X Syndrome, comprising: administering a recombinant fusion polypeptide of any one of claims 43 to 55, a polynucleotide molecule of claim 56, a vector of claim 57, or a pharmaceutical composition of claim 60, to said subject.

In one example, further comprising administration of minocycline, metformin, and/or blockers of extracellular signal-regulated kinase (ERK).

In one example, wherein said subject is a human.

In one aspect there is provided a method of treatment of a subject having or suspected of having Fragile X Syndrome, comprising:
administering a recombinant fusion polypeptide of any one of claims 1 to 13, a polynucleotide molecule of claim 14, a vector of claim 15, or a pharmaceutical composition of claim 18, to said subject and/or
administering a recombinant fusion polypeptide of any one of claims 22 to 34, a polynucleotide molecule of claim 35, a vector of claim 36, or a pharmaceutical composition of claim 39, to said subject, and/or
administering a recombinant fusion polypeptide of any one of claims 43 to 55, a polynucleotide molecule of claim 56, a vector of claim 57, or a pharmaceutical composition of claim 60, to said subject.

In one example, further comprising administration of minocycline, metformin, and/or blockers of extracellular signal-regulated kinase (ERK).

In one example, wherein said subject is a human.

### BRIEF DESCRIPTION OF THE FIGURES

Embodiments of the present disclosure will now be described, by way of example only, with reference to the attached Figures.

FIG. 1 Functional structure of FMRP. Schematic diagram of the structure of human FMRP showing the location of known functional domains and their correspondence to FMRP-tat constructs in current disclosure. FMRP is comprised of defined N- and C-terminal regions with an intervening central region. The relative span of tat-conjugate proteins delivered here as FMRP-N-tat and FMRP-C-tat are indicated schematically by dashed lines, which together comprise 76% of the total FMRP protein. Abbreviations: NDF 1 and 2: N-terminal domains that contain Tudor motifs. NLS: Nuclear Localization Signal. KH1: K-Homologous 1 motif. G266: Pathogenic mutation site. KH2: K-Homologous 2 motif. 1304: Pathogenic mutational site. NES: Nuclear Export Signal. RGG: RGG motif of RNA binding domain. S499: Serine-phosphorylation site. FMRP-N-tat: HA-FMRP(1-297)-tat. FMRP-C-tat Isoform 1: Myc-FMRP(444-632)-tat. FMRP-C-tat Isoform 17: Myc-FMRP(399-570)-tat. References: 23,26,31-33,40,52,53

FIG. 2 Structure of FMRP-tat constructs. Schematic diagrams of constructs made from a pTrc-HisA vector (Invitrogen, V360-20, Ottawa, ON) that were modified by replacing an “Xpress” epitope with the 11 aa tat (YGRKKRRQRRR; SEQ ID NO: 4) sequence and expressed in BL21 pLysS E Coli. A, Schematic diagram of the structure of an HA-tat construct alone as control. B, Schematic diagram of the myc-FMRP(444-632)-tat (FMRP-C-tat) construct for isoform 1 prepared by subcloning cDNA containing a myc sequence (EQKLISEEDL; SEQ ID NO: 6) followed by the FMRP(444-632) sequence in the pTrc-HisA-tat vector through the specific restriction enzymes site NheI and Acc65I. C, Schematic diagram of the myc-FMRP(399-570)-tat (myc-FMRP-C-tat) construct for isoform 17 prepared by subcloning cDNA containing a myc sequence (EQKLISEEDL) followed by the FMRP(399-570) sequence in the pTrc-HisA-tat vector through the specific enzymes sites NheI and Acc65I.

FIG. 3 FMRP-C-tat of isoform 1 exhibits a concentration-dependent uptake in the brain. Western blot of brain lysate prepared from P90 Fmr1 KO mice 24 hr following tail vein injection with the indicated concentrations of myc-tagged FMRP-C-tat. A detectable band is found for the 1 and 5 UM concentrations of FMRP C-tat at 25 kDa MW, a value confirmed on a blot of 10 μM FMRP-C-tat applied to the culture medium of Fmr1 CRISPR-KO HEK cells (A) and on a coomassie gel blot of the FMRP-C-tat protein (B). Purified protein of FMRP C-tat is shown as a positive control in (A). No band for FMRP C-tat is detected in brain homogenates of control Fmr1 KO mice or with vehicle-only injection.

FIG. 4 Dual immunolabeled images in male Fmr1 P60-P90 KO mice to identify the cellular distribution of myc-FMRP-C-tat (isoform 1) 24 hr post tail vein injection of vehicle or the indicated concentrations of myc-FMRP C-tat (Myc). Images were obtained in cerebellum (A) and neocortex (B) 24 hr post injection. Dashed lines in (A) delineate major cell boundaries. Abbreviations: gran, granule cell layer; PC, Purkinje cell layer; mol, molecular layer. Data indicate a concentration-dependent uptake and retention of FMRP-C-tat of individual cells in the cerebellum and cortical structures within 24 hr.

FIG. 5 FMRP-C-tat and FMRP-N-tat accumulate in the same cells in cerebellum and frontal cortex after tail vein injection. Shown are dual immunolabeled images for HA and myc 24 hr following tail vein injection of 0.5 UM HA-FMRP-N-tat (HA-N) and myc-FMRP-C-tat (isoform 1) (Myc-C) of Fmr1 P60-P90 KO mice. Images reveal uptake of both FMRP-tat constructs by Purkinje cells in cerebellum (A) and pyramidal cells of neocortex (B) 24 hr post injection. Dashed lines in (A) delineate major cell boundaries. Abbreviations: gran, granule cell layer; PC, Purkinje cell layer; mol, molecular layer.

FIG. 6 FMRP-C-tat from isoform 1 modifies Kv4.2 potassium current expressed in tsA-201 cells. Preliminary tests showing that bath applied FMRP-C-tat (100 nM) reduces the amplitude of human Kv4.2 potassium current expressed in tsA-201 cells. Whole-cell voltage mode recordings were conducted at room temperature from tsA-201 cells expressing Kv4.2 and KChIP3 with a bath solution comprised (mM) of 125 NaCl, 3.25 KCl, 1.5 CaCl2, 1.5 MgCl2, 10 HEPES, 5 TEA, 2 CsCl and 10 D-Glucose (pH adjusted to 7.3 with NaOH) and a pipette solution comprised of (mM) 110 potassium gluconate, 30 KCl, 1 EGTA, 5 HEPES, and 0.5 MgCl2, pH 7.3 via KOH, with 5 di-tris-creatine phosphate, 2 Tris-ATP, and 0.5 Na-GTP added from fresh frozen stock each day. A-type current is activated with a depolarizing step up to 60 mV from −110 mV at steps of 10 mV. Shown is the current amplitude-voltage plot in control conditions and 10 min following bath application of 100 nM FMRP-C-tat. At a voltage of 60 mV, the amplitude of A-type current is reduced by 17% with the application of FMRP-C-tat. Average values are mean±SEM (n=4). *, p<0.05.

FIG. 7 An elevated gamma power in cortices of female and male Fmr1 KO mice is reduced by FMRP-C-tat of isoform 1. EEG was recorded from frontal and auditory cortices from P60-90 female and male wild type and Fmr1 KO mice tail vein injected with vehicle or 0.8 mg/kg FMRP-C-tat. A, Superimposed mean power spectral density plots of EEG (10 log 10 (μV²·Hz⁻¹)) showing that at 3 hr post injection FMRP-C-tat reduces the elevated power in gamma frequencies in both frontal cortex (left) and auditory cortex (right) in female KO mice. Gamma frequencies are indicated right of the dashed line. A notch filter applied to remove electromagnetic noise produces a break in the records at 60 Hz. B, Mean bar graphs of the band powers for the EEG recorded from the frontal cortex showing FMRP-C-tat reduces elevated gamma power in the frontal cortex of the male KO mice at 24 hr post tail vein injection. Average values are mean±SEM, the number of mice for each group is shown in brackets. Statistical comparisons were conducted with Welch's two sample t-test. *, p<0.05; **, p<0.01; ***, p<0.001.

FIG. 8 Structure of FMRP I-tat construct. Constructs were made from a pTrc-HisA vector (Invitrogen, V360-20, Ottawa, ON) that was modified by replacing the Xpress epitope with the 11 aa tat (YGRKKRRQRRR; SEQ ID NO: 4) sequence and expressed in BL21 pLysS E Coli. Schematic diagram of myc tagged FMRP-I-tat (aa 205-399) of FMRP isoform 17 made by subcloning cDNA containing a Myc sequence (EQKLISEEDL; SEQ ID No: 6) followed by the FMRP(205-399) sequence into the pTrc-HisA-tat vector through two restriction enzymes sites NheI and Acc65I as indicated by arrows.

FIG. 9. FMRP-C-tat and FMRP-N-tat differentially rescue EEG frequencies in frontal vs auditory cortex of adult Fmr1 KO mice. Superimposed mean PSD plots of EEG (10 log 10 (μV2.Hz-1)) recorded in the frontal cortex (A-C) or auditory cortex (D-F) 72 hr after tail vein injection of vehicle in WT or Fmr1 KO (FX) mice, or FMRP-C-tat (0.8 mg/kg) or FMRP-N-tat (1.0 mg/kg) in Fmr1 KO mice. All recordings are from male P60-90 mice. The plots in (A, D) show all PSD records superimposed, with an overlay of vehicle-injected animal records with just the FMRP-C-tat data in (B, E) and FMRP-N-tat data in (C, F). A notch filter applied to remove electromagnetic noise produces a break in the records at 60 Hz. Different EEG frequency bands are identified by shades of color, with Theta, Low Gamma and High Gamma frequency ranges identified below plots. Visual inspection of data in (A-C) indicate an effect of FMRP-C-tat primarily on low gamma frequencies and FMRP-N-tat on high gamma frequencies. D-F, In the auditory cortex, FMRP-C-tat injection rescues low gamma and some aspects of high gamma frequencies (D, E) while FMRP-N-tat primarily restores high gamma frequencies to the level of WT animals injected with vehicle alone (F). Sample numbers are shown in brackets, and apply to all subsequent data sets in FIGS. 10-13.

FIG. 10. FMRP-C-tat and FMRP-N-tat differentially rescue EEG frequencies in frontal vs auditory cortex of P60-90 Fmr1 KO mice. Shown are mean bar plots of the indicated EEG frequency ranges derived from PSD plots recorded in the frontal cortex (A, B) or auditory cortex (C, D) 72 hr after tail vein injection of vehicle in WT or Fmr1 KO (FX) mice, or FMRP-C-tat (FX-C-tat, 0.8 mg/kg) or FMRP-N-tat (FX-N-tat, 1.0 mg/kg) in Fmr1 KO mice. All recordings are from male P60-90 mice and drawn from data sets shown in FIG. 9. A-D, Resting EEG of both the frontal and auditory cortex of Fmr1 KO mice exhibit a significantly lower level of theta frequencies and higher gamma frequencies, and lower delta frequencies in auditory cortex (C, D) compared to WT vehicle injected mice. Injection of FMRP-C-tat (A, C) selectively rescues theta and low gamma frequencies but not high gamma frequencies in both frontal and auditory cortex. Injection of FMRP-N-tat (B, D) selectively rescues high gamma frequencies in both frontal and auditory cortex, but not theta or low gamma frequencies. Average values are mean±SEM, with the number of samples for each group shown in FIG. 9. Statistical comparisons were conducted with Welch's two sample t-test. *, p<0.05; **, p<0.01.

FIG. 11. FMRP-C-tat and FMRP-N-tat exert long-term effects on specific EEG frequencies in the frontal cortex of Fmr1 KO mice. Shown are mean scatter plots of the indicated EEG frequency ranges derived from PSD plots recorded in the frontal cortex at the indicated times after tail vein injection of vehicle in WT or Fmr1 KO (FX) mice (A-F), or FMRP-C-tat (FX-C-tat, 0.8 mg/kg) (A-C) or FMRP-N-tat (FX-N-tat, 1.0 mg/kg) (D-F) in Fmr1 KO mice. All recordings are from male P60-90 mice and drawn from data sets shown in FIG. 7. A-C, FMRP-C-tat injections rescue theta (A) and low gamma (B) frequencies within 2 hr injection, but not high gamma frequencies (C). Both theta and low gamma frequency rescue by FMRP-C-tat is intact 3 days following a single injection. D-F, FMRP-N-tat injection does not rescue either theta (D) or low gamma (E) frequencies, but achieves full rescue of high gamma frequencies (F) within 2 hr that persists throughout a 72 hr recording period. Average values are mean±SEM, with the number of samples for each group shown in FIG. 9.

FIG. 12. FMRP-C-tat and FMRP-N-tat exert long-term effects on specific EEG frequencies in the auditory cortex of Fmr1 KO mice. Shown are mean scatter plots of the indicated EEG frequency ranges derived from PSD plots recorded in the auditory cortex at the indicated times after tail vein injection of vehicle in WT or Fmr1 KO (FX) mice (A-F), or FMRP-C-tat (FX-C-tat, 0.8 mg/kg) (A-C) or FMRP-N-tat (FX-N-tat, 1.0 mg/kg) (D-F) in Fmr1 KO mice. All recordings are from male P60-90 mice and drawn from data sets shown in FIG. 9. A-C, FMRP-C-tat injections rescue theta (A) and low gamma (B) frequencies within 2 hr injection, but with less efficacy for high gamma frequencies (C). Both theta and low gamma frequency rescue by FMRP-C-tat is intact 3 days following a single injection. D-F, FMRP-N-tat injection does not rescue theta (D) frequency, has variable rescue of low gamma (E) frequencies, but a more convincing rescue of high gamma frequencies (F) within 2 hr and throughout a 72 hr recording period. Average values are mean±SEM, with the number of samples for each group shown in FIG. 9.

FIG. 13. FMRP-C-tat and FMRP-N-tat exert complementary rescue of select cortical EEG frequencies in Fmr1 KO mice for at least 3 days. A, C, Summary mean bar plots of the indicated EEG frequency ranges comparing the rescue effects of FMRP-tat peptides in the frontal cortex (A) and auditory cortex (C). Data represent EEG recorded 72 hr after tail vein injection of vehicle in WT or Fmr1 KO (FX) mice, or FMRP-C-tat (FX-C-tat, 0.8 mg/kg) or FMRP-N-tat (FX-N-tat, 1.0 mg/kg) in Fmr1 KO mice. FMRP-C-tat acts to rescue theta frequencies and low gamma frequencies in both frontal (A) and auditory cortex (C) but not high gamma frequencies. In contrast, FMRP-N-tat injection does not rescue theta or low gamma frequencies in either frontal or auditory cortex, with actions focused instead on high gamma frequencies primarily in frontal cortex. B, D, Superimposed mean PSD plots of the frequencies of EEG rescued by FMRP-C-tat or FMRP-N-tat injections in the frontal cortex (B) or auditory cortex (D). PSD representations were calculated through subtraction of mean PSD values for drug treated vs vehicle injected Fmr1 KO mice and presented on the same scale. A complementarity of rescue effects by FMRP-C-tat and FMRP-N-tat in the frontal cortex is apparent in almost mirrored representations of EEG frequencies rescued by 72 hr after injections (B). D, FMRP-C-tat and FMRP-N-tat have a similar pattern of effects on EEG frequencies in the auditory cortex as in the frontal cortex, but of lower relative magnitude exerted over a broader range of frequencies than in frontal cortex.

### DETAILED DESCRIPTION

Generally, the present disclosure relates to the treatment of Fragile X syndrome.

Fragile X syndrome (FXS) refers to a genetic disease associated with and/or caused by a defect of the expression of the FMR1 gene and/or of the activity of the FMR1-encoded polypeptide, FMRP.

In some examples, signs and symptoms of FXS may fall into five categories: intelligence and learning; physical, social and emotional, speech and language and sensory disorders commonly associated or sharing features with Fragile X.

For example, individuals with FXS may have impaired intellectual functioning, social anxiety, language difficulties and sensitivity to certain sensations.

Cognitive disorders may include, but are not limited to, the group of disorders in which a dysfunction/impairment of mental processing constitutes the core symptomatology. Cognitive disorders include neurogenetic cognitive disorders or behavioral cognitive disorders

Cognitive disorders may include, but are not limited to, developmental disorders, attention deficit hyperactivity disorder (ADHD), autism spectrum disorders, Alzheimers disease, schizophrenia and cerebrovascular disease.

Autism spectrum disorders and autistic symptoms are commonly associated with individuals with Fragile X syndrome. Signs and symptoms of autism may include, but are not limited to, significant language delays, social and communication challenges, and unusual behaviors and interests. Individuals with autistic disorder may also have intellectual disability.

Methods of assessment of Fragile X Syndrome in a subject are known. Accordingly, methods of assessing efficacy of treatment of Fragile X Syndrome in a subject are known.

In one aspect, there is provided a recombinant fusion polypeptide, comprising: a FMRP(444-632) polypeptide and a cell penetrating polypeptide, or fragment or variant thereof.

In one aspect, there is provided a method of treatment of a subject having or suspected of having Fragile X Syndrome, comprising: administering to said subject a recombinant fusion polypeptide, comprising: a FMRP(444-632) polypeptide and a cell penetrating polypeptide, or fragment or variant there of

In one aspect, there is provided recombinant fusion polypeptide, comprising: a FMRP(399-570) polypeptide and a cell penetrating polypeptide, or fragment or variant thereof.

In one aspect, there is provided a method of treatment of a subject having or suspected of having Fragile X Syndrome, comprising: administering to said subject a recombinant fusion polypeptide, comprising: a FMRP(399-570) polypeptide and a cell penetrating polypeptide, or fragment or variant thereof.

In one aspect, there is provided a recombinant fusion polypeptide, comprising: a FMRP(205-399) polypeptide and a cell penetrating polypeptide, or fragment or variant thereof.

An one aspect, there is provided a method of treatment of a subject having or suspected of having Fragile X Syndrome, comprising: administering to said subject a recombinant fusion polypeptide, comprising: a FMRP(205-399) polypeptide and a cell penetrating polypeptide, or fragment or variant thereof.

The term “subject” or “patient” are used synonymously, and as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. The subject may be an infant, a child, an adult, or elderly. In a specific example, the subject is a human.

As used herein, “treatment” refers to any manner in which one or more of the symptoms of a disorder, such as FXS, are ameliorated or otherwise beneficially altered. Thus, the terms “treating” or “treatment” of a disorder as used herein includes: reverting the disorder, i.e., causing regression of the disorder or its clinical symptoms wholly or partially; preventing the disorder, i.e. causing the clinical symptoms of the disorder not to develop in a subject that can be exposed to or predisposed to the disorder but does not yet experience or display symptoms of the disorder; inhibiting the disorder, i.e., arresting or reducing the development of the disorder or its clinical symptoms; attenuating the disorder, i.e., weakening or reducing the severity or duration of a disorder or its clinical symptoms; or relieving the disorder, i.e., causing regression of the disorder or its clinical symptoms. Further, amelioration of the symptoms of a particular disorder by administration of a particular composition refers to any lessening, whether permanent or temporary, lasting or transient that can be attributed to or associated with administration of the disclosed recombinant fusions polypeptides, compounds, compositions, etc.

As used herein, the terms “polypeptide”, “peptide” and “protein,” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

In some examples, a “fusion polypeptide” or “fusion protein” is a recombinant protein of two or more polypeptides which are joined by a peptide bond. In some examples, the two or more polypeptides may be joined by a linker.

The fusion polypeptide may include variants of a fusion polypeptide. In some examples, a variant of a fusion polypeptide refers to fusion polypeptides having different sequence from wild type amino acid sequence. For examples, a variant fusion polypeptide may have deletions, insertions, non-conservative or conservative substitutions of at least one amino acid residue, or combinations thereof.

In some examples, the recombinant polypeptide is a variant of the polypeptide of the recombinant fusion protein

In another example, there is provided a recombinant fusion polypeptide, a tat-conjugated FMRP “intermediate” protein sequence (FMRP-I-tat), designed to match that of isoform 17 of the human brain, or fragment or variant thereof.

The intermediate (I) region (central domain) includes the KH1 and KH2 domain and most definitions of a NES site.

In one example, the FMRP(205-399) polypeptide is

(SEQ ID NO: 1)
MSKQLESSRQLASRFHEQFIVREDLMGLAIGTHGANIQQARKVPGVTAIDLDEDTCTFHI
YGEDQDAVKKARSFLEFAEDVIQVPRNLVGKVIGKNGKLIQEIVDKSGVVRVRIEAENEK
NVPQEEVLVASSVVAGESQKPELKAWQGMVPFVFVGTKDSIANATVLLDYHLNYLKEVD
QLRLERLQIDEQLRQI.

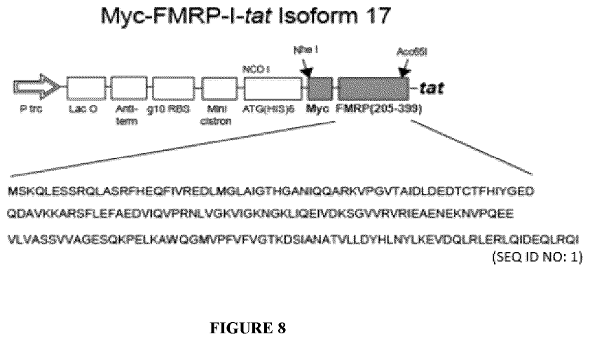

FIG. 8A depicts a structure of FMRP I-tat construct.

In one example, the FMRP(444-632) polypeptide is:

(SEQ ID NO: 7)
MGASSRPPPNRTDKEKSYVTDDGQGMGRGSRPYRNRGHGRRGPGYTSGTNSEASNA
SETESDHRDELSDWSLAPTEEERESFLRRGDGRRRGGGGRGQGGRGRGGGFKGND
DHSRTDNRPRNPREAKGRTTDGSLQIRVDCNNERSVHTKTLQNTSSEGSRLRTGKDRN
QKKEKPDSVDGQQPLVNGVP.

In one example, the FMRP(399-570) polypeptide is:

(SEQ ID NO: 8)
MGASSRPPPNRTDKEKSYVTDDGQGMGRGSRPYRNRGHGRRGPGYTSGTNSEASNA
SETESDHRDELSDWSLAPTEEERESFLRRGDGRRRGGGGRGQGGRGRGGGFKGND
DHSRTDNRPRNPREAKGRTTDGSLQNTSSEGSRLRTGKDRNQKKEKPDSVDGQQPLV
NGVP.

In some examples, the “variant” as it relates to polypeptides refers to polypeptides having an amino acid sequence that is at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% or greater identical to the parental amino acid sequence.

The fusion polypeptide and/or variants thereof may be chemically synthesized or produced by gene recombination, and it may be produced by transforming host cells using a recombinant vector and separating and purifying expressed protein.

The term “recombinant” as used herein refers to a non-naturally occurring nucleic acid, nucleic acid construct, or polypeptide. Such non-naturally occurring nucleic acids can include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a “fusion protein” (e.g., a protein or polypeptide formed from the combination of two different proteins or protein fragments)), the combination of a nucleic acid encoding a polypeptide to a promoter sequence, where the coding sequence and promoter sequence are from different sources or otherwise do not typically occur together naturally (e.g., a nucleic acid and a constitutive promoter etc.). Recombinant also refers to the polypeptide encoded by the recombinant nucleic acid. Recombinant may also refer to a polypeptide or polynucleotide, for example, that is no longer in its natural environment

Also provided herein are recombinant polynucleotides that may encode a recombinant fusion polypeptide described herein. In one example, a polynucleotide encodes a polypeptide comprising or consisting of a recombinant fusion protein.

A polynucleotide encoding the fusion protein may be codon optimized for efficient translation into a polypeptide in the eukaryotic cell or animal of interest.

As used herein the terms “polynucleotide” and “nucleic acid’ refer to two or more nucleosides that are covalently linked together. The polynucleotide may be wholly comprised ribonucleosides (i.e., an RNA), wholly comprised of 2′ deoxyribonucleotides (i.e., a DNA) or mixtures of ribo- and 2′ deoxyribonucleosides. Typically nucleosides will be linked together via standard phosphodiester linkages. However, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine, and cytosine), it may include one or more modified and/or synthetic nucleobases (e.g., inosine, xanthine, hypoxanthine, etc.). Polynucleotide includes, but is not limited to chemically, enzymatically, or metabolically modified forms.

In some examples there is provided a polynucleotide molecule comprising or consisting of a sequence that encodes a recombinant fusion polypeptide comprising a FMRP(444-632) polypeptide and a cell penetrating polypeptide, or fragment or variant thereof.

In some examples there is provided a polynucleotide molecule comprising or consisting of a sequence that encodes a recombinant fusion polypeptide, comprising: a FMRP(399-570) polypeptide and a cell penetrating polypeptide, or fragment or variant thereof.

In some examples there is provided a polynucleotide molecule comprising or consisting of a recombinant fusion polypeptide, comprising: a FMRP(205-399) polypeptide and a cell penetrating polypeptide, or fragment or variant thereof.

In some examples, there is provided a variant of a polynucleotide molecule comprising or consisting of a sequence that encodes a recombinant fusion polypeptide comprising a FMRP(444-632) polypeptide and a cell penetrating polypeptide, or fragment there of

In some examples, there is provided a variant of a polynucleotide molecule comprising or consisting of a sequence that encodes a recombinant fusion polypeptide, comprising: a FMRP(399-570) polypeptide and a cell penetrating polypeptide, or fragment thereof.

In some examples, there is provided a variant of a polynucleotide molecule comprising or consisting of a sequence that encodes a recombinant fusion polypeptide, comprising: a FMRP(205-399) polypeptide and a cell penetrating polypeptide, or fragment thereof.

As used herein, the terms “polynucleotide variant” and the like refer to polynucleotides displaying substantial sequence identity with a reference polynucleotide sequence or polynucleotides that hybridize with a reference sequence under, for example, stringent conditions. These terms may include polynucleotides in which one or more nucleotides have been added or deleted, or replaced with different nucleotides compared to a reference polynucleotide. It will be understood that that certain alterations inclusive of mutations, additions, deletions and substitutions can be made to a reference polynucleotide whereby the altered polynucleotide retains the biological function or activity of the reference polynucleotide.

In some examples, the “variant” as it relates to polynucleotides refers to polynucleotides having a nucleotide sequence that is at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% or greater identical to the parental polynucleotide sequence.

In some examples, the recombinant fusion polypeptide is encoded by a variant fusion polynucleotide that binds under high hybridization stringency to the fusion polynucleotide.

As used herein the term “hybridization stringency” refers to hybridization conditions, such as washing conditions, in the hybridization of nucleic acids. Generally, hybridization reactions are performed under conditions of lower stringency, followed by washes of varying but higher stringency.

Under high stringency conditions, a polynucleotide with higher identity is expected to hybridize efficiently at higher temperatures, though multiple factors are involved in hybridization stringency including temperature, probe concentration, probe length, ionic strength, time, salt concentration and others, and a person skilled in the art may appropriately select these factors to achieve similar stringency.

In some examples “high stringency” may refer to the use of a hybridization or wash solution comprising 10 mM phosphate buffer, pH 7.0, at a range of about 45-55° C. In some examples, “moderate stringency” may refer to the use of 10 mM phosphate buffer, pH 7.0, with a salt concentration of about 0.1 to 0.5 M NaCl, at a temperature of between about 30 to 45° C. In some examples, “low stringency” may refer to the use of about 10 mM phosphate buffer at about pH 7.0, 1.0 M NaCl at room temperature. Low stringency buffers may also include 10 mM MgCl2. It will be understood that that many factors, such as temperature, salt and inclusion of other components such as formamide, affect the stringency of hybridization.

In one example, there is provided a vector comprising a polynucleotide as described herein.

The term “vector” is used herein to refer to a nucleic acid molecule capable of transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. The polypeptide or polynucleotide may be isolated.

By an “isolated” polypeptide, polynucleotide, fragment, variant, or derivative thereof is intended a polypeptide or polynucleotide that is not in its natural milieu. No particular level of purification is required. For example, an isolated polypeptide can be removed from its native or natural environment. Recombinantly produced polypeptides expressed in host cells are considered isolated for purposed of the invention, as are native or recombinant polypeptides which have been separated, fractionated, or partially or substantially purified by any suitable technique.

It will be appreciated that in some example, the host cell (also referred to as a cell) may be eukaryotic or prokaryotic.

In some examples, the isolated polypeptide or polypeptide may be purified.

As used herein, “pure” or “purified” means an object species is the predominant species present (i.e., on a molar and/or mass basis, it is more abundant than any other individual species, apart from water, solvents, buffers, or other common components of an aqueous system in the composition), and, in some embodiments, a purified fraction is a composition wherein the object species comprises at least about 50% (on a molar basis) of all macromolecular species present. Generally, a “substantially pure” composition will comprise more than about 80% of all macromolecular species present in the composition, in some embodiments more than about 85%, more than about 90%, more than about 95%, or more than about 99%. In some embodiments, the object species is purified to essential homogeneity (contaminant species cannot be detected in the composition by conventional detection methods) wherein the composition consists essentially of a single macromolecular species.

In one example, the recombinant fusion polypeptide(s) described herein may be used for administration to a subject.

Administration may be in vitro, ex vivo or in vivo.

Administering may also be performed, for example, once, a plurality of times, and/or over one or more extended periods.

Administration may be by any suitable means.

In some examples, the recombinant polypeptides are formulated as a pharmaceutical composition, which is pharmaceutically acceptable.

The phrase “pharmaceutically acceptable” indicates that the substance or composition must be compatible chemically and/or toxicologically, with the other ingredients comprising a formulation, and/or the subject being treated.

The recombinant fusion polypeptide may be formulated with pharmaceutically acceptable carriers, excipients or diluents.

Pharmaceutically acceptable carriers include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Compositions as described herein may be sterilized by conventional methods and/or lyophilized.

Routes of administration include, but are not limited to, injection (subcutaneous, intravenous, parenterally, intraperitoneally, intrathecal), oral, inhalation, rectal and transdermal. The pharmaceutical compositions may be given by forms suitable for each administration route. For example, these compositions are administered in tablets or capsule form, by injection, inhalation, eye lotion, ointment, suppository, etc. administration by injection, infusion or inhalation; topical by lotion or ointment; and rectal by suppositories. In some embodiments, the pharmaceutical compositions may be administered by oral administration. The injection can be bolus or can be continuous infusion. Depending on the route of administration, a recombinant fusion polypeptide described herein can be coated with or disposed in a selected material to protect it from natural conditions which may detrimentally affect its ability to perform its intended function. A recombinant fusion polypeptide, compound or composition described herein can be administered alone, or in conjunction with either another agent as described above or with a pharmaceutically-acceptable carrier, or both. A recombinant fusion polypeptide, compound or composition described herein can be administered prior to the administration of the other agent, simultaneously with the agent, or after the administration of the agent. Furthermore, a recombinant fusion polypeptide, compound described herein can also be administered in a pro-drug form which is converted into its active metabolite, or more active metabolite in vivo.

In some examples, there is further provided co-administration or use with a second agent. In some examples, the second agent may be minocycline, metformin, and/or blockers of extracellular signal-regulated kinase (ERK) such as lovastatin and related compounds.

Methods of the invention are conveniently practiced by providing the recombinant fusion polypeptide, compounds and/or compositions used in such methods in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

### Example 1

Fragile X Syndrome (FXS) is the leading monogenic factor behind behavioral dysfunction in autism spectrum disorders (ASD) in aspects of cognition, communication, and socialization¹⁻⁵. FXS results from disrupted expression of Fragile X Messenger Ribonucleoprotein (FMRP) that reduces neural circuit function through effects that span from the level of protein translation to circuit level output^{1,6-10}. Many of the ASD-related behaviours are known to be associated with activity in the frontal cortex¹¹⁻¹⁶. The fact that FXS stems from the loss of a single protein raises the possibility that reintroducing FMRP would offset the primary factor underlying this genetic disorder. Indeed, it was recently reported that increasing FMRP levels by only 5% is sufficient to normalize a hyperactive neuronal phenotype in human FXS iPSC cells¹⁷. An early study tested a full-length FMRP conjugated to the HIV-1 trans-activator of transcription (tat) peptide to facilitate its passage across the blood-brain barrier (BBB)¹⁸. However, they reported toxicity on cultured fibroblasts>7.5 nM, quelling expectations that a tat-conjugate approach could be used to treat FXS¹⁸. We linked tat to an N-terminal segment (aa 1-297) of FMRP(FMRP-N-tat) to reintroduce by tail vein injection. FMRP-N-tat proved to have no detectable toxic effects and rapidly crossed the BBB to distribute across the CNS, restore levels of protein translation, rescue synaptic plasticity, and reduce hyperactivity in the Open Field Test for up to 24 hr⁸.

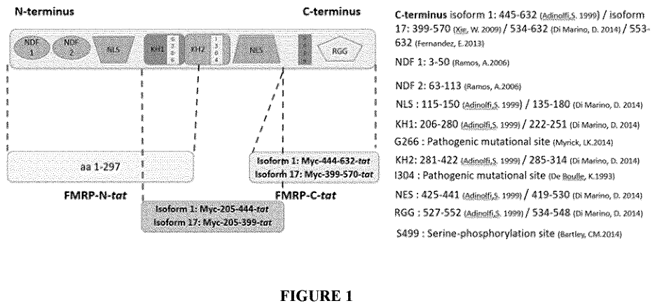

While not wishing to be bound by theory, it is believed the effects mediated by FMRP-N-tat injections reflect interactions involving structural regions of the FMRP N-terminus that have defined functions, including N-terminal Domain of FMRP(NDF) domains, a nuclear localization sequence (NLS), and a KH1 domain for binding to numerous mRNA for translational control^{1, 6, 10, 19-24}. By comparison, the C-terminal region of FMRP contains an RGG box domain for RNA binding and the S499 site for phosphorylation to regulate translation^{6, 25-28} (FIG. 1). The C-terminal region was already shown to bind to Cav2.2 (N-type) calcium channels to decrease its membrane expression in dorsal root ganglion²⁹.

Designing tat-conjugates: While the structure and function of the FMRP molecule has been thoroughly studied, the exact sequences proposed to mediate key functions differ between studies³⁰; ³¹⁻³³. The most studied isoform for FMRP-N-tat(1-297) was developed from studies on isoform 1⁸. We have first tested a FMRP C-terminal sequence (as defined by Adinolfi et al.^{31, 34}) from a.a. 444-632 of isoform 1 (hereafter referred to as FMRP-C-tat), a region with 88% homology between mouse and human that contains an S499 phosphorylation site to regulate translation²⁵⁻²⁷ and an RGG box domain^{6, 28, 35, 36} (FIG. 1). Recent work has also highlighted the fact that there are 26 isoforms of FMRP protein in human tissues with isoform 17 (54% of transcripts) and isoform 7 (26% of transcripts) most abundantly expressed in the brain³⁷⁻³⁹. We note that the sequence of isoform 1 FMRP(1-297) is identical to that of isoform 17. Given the recognition and prevalence of isoform 17 we will also test the FMRP C-terminal sequence inherent to isoform 17 (FMRP(399-570)-tat) that is comprised of 17 fewer amino acids but contains the same functional domains as that found in isoform 1. We recognize that these C-terminal constructs lack the amino acid sequence that contains a KH2 domain^{21, 32, 40, 41} and most definitions of a NES site^{30, 31, 33, 42}. Nevertheless, the chosen length of C-terminal regions have several identified functions and comprises ~29% of the full FMRP sequence.

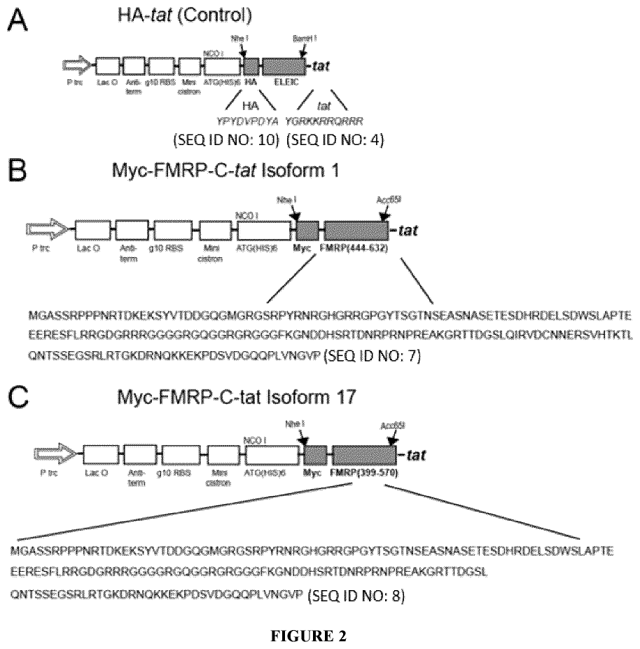

The procedures used to generate HA-FMRP-N-tat (human FMRP isotype 1 used for DNA construction, NM_002024.5) are published⁸, with a similar process used to prepare FMRP-C-tat isoform 1 (FMRP-C-tat). Specifically, we conjugated the 11 a.a. cell penetrating moiety tat(YGRKKRRQRRR; SEQ ID NO:4) to an HA tag that can be located immunocytochemically for control tests (FIG. 2A) and to FMRP(444-632) to create FMRP-C-tat isoform 1 with a myc tag at the N-terminal aspect (FIG. 2B). A similar process will be followed to generate a myc-tagged FMRP-(399-570)-tat construct from FMRP isoform 17 (FIG. 2C).

### General Methods

Preparations: Breeding colonies of Fmr1 KO and WT mice on the FVB.129P2 background were used. All procedures adhered to the Policies of the Canadian Council on Animal Care and the University of Calgary Animal Care Committee. tsA-201 cells were cultured according to established procedures 8 and channel cDNA transfected for expression and whole-cell patch clamp recording at room temperature as previously described^{8,43}.

FMRP-C-tat preparation: FMRP-C-tat(isoform 1) was prepared from a pTrc-HisA vector (Invitrogen, V360-20, Ottawa, ON) originally modified to create FMRP-N-tat by replacing an “Xpress” epitope with the 11 aa tat(YGRKKRRQRRR; SEQ ID NO: 4) sequence and expressed in BL21 pLysS E Coli. FMRP-C-tat was purified with Ni-NTA Fast Start Kit (Qiagen) and stored in elute buffer at 2 μg/μl at −80° C., and the concentration rechecked upon thawing using a Bradford Protein Assay (Bio-Rad). Myc- or HA-tagged FMRP-C-tat protein was tail vein injected at concentrations of 0.24-1.6 mg/kg in 0.9% NaCl, 200 μl volume, or applied directly to culture medium of Fmr1 CRISPR KO HEK cells at 100 nM.

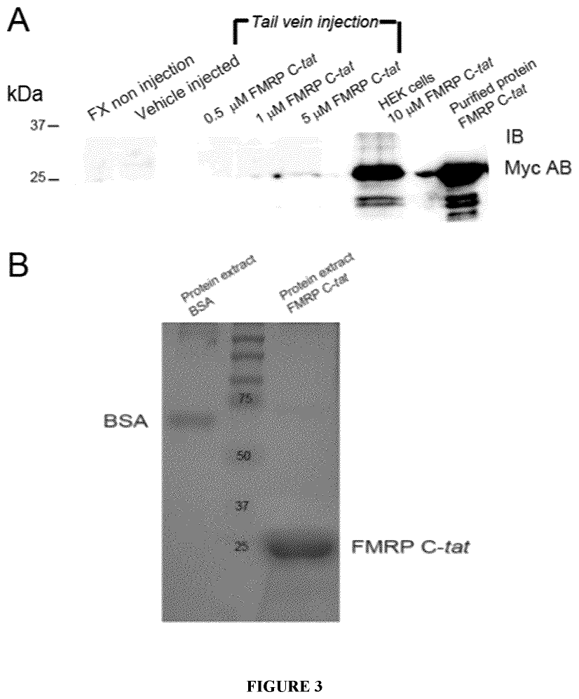

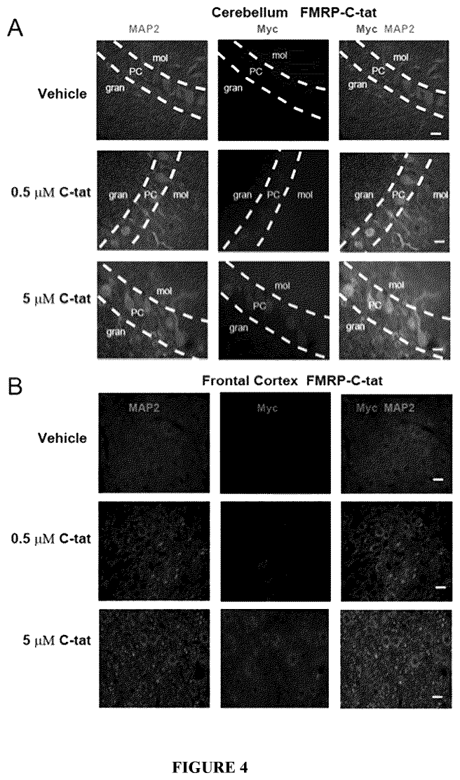

FMRP-C-tat conjugate detection: Myc-FMRP-C-tat was detected 24 hr following tail vein injection into P60-P90 Fmr1 KO mice on Western blots from brain lysates (FIG. 3A) or in fixed tissue sections (FIG. 4). FMRP-C-tat distribution was also detected 24 hr following application of 100 nM myc-FMRP-C-tat on Fmr1 CRISPR KO HEK cells (FIG. 3A).

Tissue sections: Standard operating procedures were used to euthanize animals by isoflurane inhalation and perform cardiac perfusion of 4% paraformaldehyde to prepare free floating tissue sections for immunocytochemistry, as per Zhan et al. (2020). Tissue sections were dual labeled using anti-myc (1:2000, CST mAb #2276) for Myc-FMRP-C-tat detection and anti-MAP2 (1:1000, AbCaM mAb #5392) for neuron specific protein marker detection for overnight at 4° C., and after washes and primary antibodies fluorophore labeled using goat anti mouse and goat anti chicken secondary antibodies (1:1000) for 1 hr at room temperature. Images were obtained on a Zeiss fluorescence microscope equipped with Colibri LED light source and Apotome structured illumination.

Western blots: Western blots were obtained using tissue lysates prepared from brains of P90 Fmr1 KO mice and Fmr1 CRISPR KO HEK cells (FIG. 3A). Both samples were homogenized in a lysis buffer containing (mM): 150 NaCl, 50 Tris, 2.5 EGTA, 1% NP-40, pH 7.5, phosphatase inhibitor (P5726, Sigma-Aldrich) and proteinase inhibitor (04693124001, Roche). The homogenates were then centrifuged at 13000 g for 10 min at 4° C. Supernatants were collected and concentrations were measured using the Bradford Protein Assay (Bio-Rad). Brain and Fmr1 CRISPR KO HEK cells lysates were loaded onto 12% Tris-glycine gel and resolved using SDS-PAGE, with 30 μg total protein for Fmr1 KO mice injected with FMRP-C-tat or vehicle, 50 μg total protein for Fmr1 CRISPR KO HEK cells after application of myc-FMRP-C-tat, and 40 μg or 5 μg for purified FMRP-C-tat as a positive control. Samples were transferred to 0.2 μm PVDF membrane (Millipore) and probed with primary anti-Myc antibody (1:1000, CST mAb #2276) overnight at 4° C., followed by goat anti-mouse HRP-conjugated secondary antibody (1:3000, Invitrogen 62-6520) for 1 hr at room temperature. Positive controls consisted of purified FMRP C-tat protein and negative control lysates derived from untreated Fmr1 KO mice and Fmr1 KO mice or Fmr1 CRISPR KO HEK cells injected or treated with vehicle alone. Coomassie stained blots were also prepared from purified FMRP C-tat protein and Bovine Serum Albumin (BSA) protein as positive control and were loaded onto 12% Tris-glycine gel and stained using Coomassie blue staining (FIG. 3B). Western blots were imaged using a chemiluminescent western blot detection system (Li-Cor Biosciences).

EEG: EEG activity of frontal cortex was recorded in P60-90 female WT and Fmr1 KO mice using surface electrodes mounted as 4 screws (2.54 mm long, 0.6 mm in diameter with wire lead, Pinnacle Technology) positioned in the skull with following coordinates (relative to Bregma, anterior/posterior, medial/lateral, in mm): Reference (−3.5, −3.5), Ground (+2.0, +1.0), frontal recording (+2.0, −1.0), auditory recording (−1.6, +3.5). Surgery was performed under isoflurane anesthesia on a stereotaxic frame (Kopf Model 900, Tujunga, CA) to place 4 surface electrodes that were then soldered onto a connector cap that was fixed to the skull with dental cement. After 1 week recovery mice were connected under isoflurane anesthesia to the head connector of a Sirenia EEG machine (Pinnacle Technologies Inc., Kansas, USA) to record EEG and video of movement using Pinnacle software. Recordings were obtained continuously for 3 days in WT and Fmr1 KO mice with one-time administration of either vehicle or FMRP-C-tat(0.24-1.6 mg/kg) through tail-vein injection. During post hoc analysis 5 min of recordings were identified during which mice were active as confirmed with video recording and EEG signal extracted. Power spectral analyses were then conducted in Matlab using custom made scripts.

### Results

The FMRP C-terminal sequences itself for isoform 1 is from a.a. 444-632, the total is 189 a.a., and for isoform 17 is from a.a. 399-570, the total is 172 a.a. Those sequences are inserted in a PtrC His vector, tagged at the N-terminal end with myc as an antigen that can be detected immunocytochemically, and an 11 a.a. tat sequence at the C-terminal end to facilitate transport of the molecule across the blood-brain barrier (BBB) (FIG. 2). All together with some a.a. from PtrC His vector (14 a.a.), Myc sequences (10 a.a.), the short linkage sequences (5 a.a.), and tat sequences (11 a.a.) makes the total of the C-tat sequence is 229 a.a. long and approximately 25 kDa for isoform 1, and 212 a.a. long and approximately 23 kDa for isoform 17, which is well within the size that tat compound can function as a cell-permeable molecule.

### Structure of FMRP-C-tat

### Isoform 1:

In one aspect there is described a fusion polypeptide consisting of a pTrc vector containing a His tag, Myc tag, FMRP(444-632), and tat sequence, together referred to here as FMRP-C-tat:

(SEQ ID NO: 2)
MGGSHHHHHHGMASEQKLISEEDLMGASSRPPPNRTDKEKSYVTDDG
QGMGRGSRPYRNRGHGRRGPGYTSGTNSEASNASETESDHRDELSDWSLAPTEEER
ESFLRRGDGRRRGGGGRGQGGRGRGGGFKGNDDHSRTDNRPRNPREAKGRTTDGS
LQIRVDCNNERSVHTKTLQNTSSEGSRLRTGKDRNQKKEKPDSVDGQQPLVNGVPGS
GGGYGRKKRRQRRR (FIG. 2A).

### Isoform 17:

In one aspect there is described a fusion polypeptide consisting of a pTrc vector containing a His tag, Myc tag, FMRP(399-570), and tat sequence, together referred to here as FMRP-C-tat:

(SEQ ID NO: 3)
MGGSHHHHHHGMASEQKLISEEDLMGASSRPPPNRTDKEKSYVTDDG
QGMGRGSRPYRNRGHGRRGPGYTSGTNSEASNASETESDHRDELSDWSLAPTEEER
ESFLRRGDGRRRGGGGRGQGGRGRGGGFKGNDDHSRTDNRPRNPREAKGRTTDGS
LQNTSSEGSRLRTGKDRNQKKEKPDSVDGQQPLVNGVPGSGGGYGRKKRRQRRR
(FIG. 2B).

Western blots: Our initial work using immunocytochemistry against an HA-FMRP-N-tat established its ability to cross the BBB within 30 min after tail vein injections of 0.2-1.0 mg/kg and distributed widely over the cerebellar-cortical axis. HA immunolabeled neurons remained detectable for at least 48 hr 8. Western Blot detection of FMRP-N-tat was possible in brain lysates by enriching the sample through initial immunoprecipitation of HA and then detection by an N-terminal antibody.

We repeated these tests using tail vein injections of 0.8-8 mg/kg FMRP-C-tat or vehicle alone in P90 Fmr1 KO mice (200 μl volume) and then processed the brains 24 hr later for Western blot detection (FIG. 3A). Using a protein loading concentration of 30 μg, an anti-myc antibody identified a clear single band at ~25 kDa MW for lysates derived from Fmr1 KO mice injected with either 1.6 mg/kg or 8 mg/kg but not 0.8 mg/kg myc-FMRP-C-tat(FIG. 3A), which corresponds to the value predicted for the combined mass of a myc-FMRP-C-tat construct. No band was detected in lysates from Fmr1 KO mice with or without injection of vehicle alone but a band of ~25 kDa was found for a lane loaded at 40 μg of purified FMRP-C-tat protein (FIG. 3A). Exposure of CRISPR-FMRP KO HEK cells to 10 UM FMRP-C-tat for 24 hr was also detected on Western blot (FIG. 3A). A single band of ~25 kDa MW detected on a 12% Tris-glycine gel labeled by Coomassie blue staining (FIG. 3B), indicating no dimerization of the C-terminal fragment as found for that of the FMRP(1-297) fragment used in FMRP-N-tat.

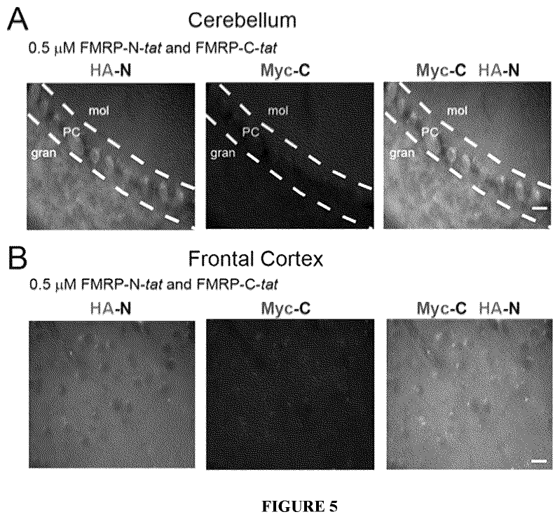

Immunolabel: Previous work determined that HA-tagged FMRP-N-tat could be detected histologically in Fmr1 KO mice in brain tissue sections with an HA antibody at times as short as 30 min after tail vein injections 8. In that case, HA immunolabel was detected in both Purkinje and granule cells of cerebellum at even low doses of FMRP-N-tat injection (0.2 mg/kg), with additional labeling in neocortical and hippocampal tissues that increased in fluorescent intensity over 24 hr time. To test the ability for myc-FMRP-C-tat to cross the BBB we tested for myc labeling in brain tissue sections prepared 24 hr after tail vein injection of 0.8 mg/kg or 8 mg/kg Myc-FMRP-C-tat in P30-P60 Fmr1 KO mice. Fixed tissue sections of 50 μm thickness were processed for labeling using an anti-myc antibody and anti-MAP2 antibody as a general structural marker. While no labeling for myc was detected in sections prepared from mice injected with vehicle alone (FIG. 4A, B), immunolabeling in cerebellum revealed a concentration-dependent increase in label within the somatic region of both Purkinje and granule cells (FIG. 4A). Similar results were obtained in neocortical tissue, where tissue sections indicated a concentration-dependent increase in myc immunolabeling in cortical pyramidal cell bodies (FIG. 4B). This is important in verifying that FMRP-C-tat successfully crossed the blood-brain-barrier after tail vein injection, with a concentration-dependent increase in immunolabel detection following uptake by principal neurons in both cerebellum and neocortex 24 hr later. We also tested the ability for HA-FMRP-N- and C-tat to both be taken up by the same cells after co-injection. Brain tissue sections were prepared 24 hr after tail vein injection of 1 mg/kg HA-FMRP-N-tat and 0.8 mg/kg Myc-FMRP-C-tat(calculated blood concentration both at 0.5 μM) in P30-P60 Fmr1 KO mice. Fixed tissue sections of 50 μm thickness were processed for labeling using an anti-myc antibody and anti-HA antibody. Immunolabeling revealed both HA and Myc labels for FMRP N-tat and FMRP-C-tat in cerebellar Purkinje cells (FIG. 5A) and cortical pyramidal cells (FIG. 5B). This is important in indicating that the effects of the N terminal and C-terminal regions of FMRP delivered as tat-conjugated peptides can act within the same cells to potentially produce synergistic effects on the symptoms of FXS.

Kv4 recordings: Our previous work showed that FMRP is important to a form of memory formation at the level of sensory input to cerebellum, measured in terms of long-term potentiation (LTP) of mossy fiber input to cerebellar granule cells^{8,43}. Further work showed that FMRP is a constitutive element of a complex formed between a class of calcium channels (Cav3 family) and potassium channels (Kv4 family) that is required to evoke LTP at this important synaptic junction⁸. Direct infusion of an active fragment of the FMRP N terminal (aa 1-297) into granule cells proved to rescue LTP at this synapse within 10 min in vitro. Key to this process was the ability for FMRP-N-tat to enable a synaptically evoked “leftward” (hyperpolarizing) shift in the measured voltage for inactivation (Vh) of Kv4 potassium channels in granule cells.

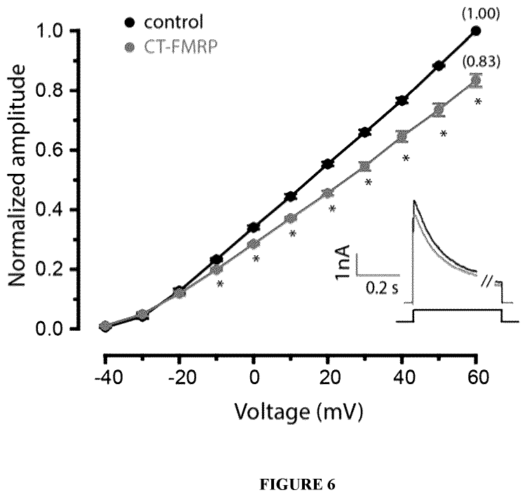

To test the potential for FMRP-C-tat to similarly affect the function of the Cav3-Kv4 complex we first tested the effects of this compound on Kv4.2 channels coexpressed with the calcium sensor KChIP3 in tsA-201 cells to obtain whole-cell recordings at room temperature. A-type current was activated with a depolarizing step to 60 mV from −110 mV in 10 mV increments (FIG. 6). The amplitude of Kv4.2 current was reduced by 17% (0.83±0.02, p<0.05) as measured for the 60 mV voltage step 10 mins after bath application of 100 nM FMRP-C-tat. Further analysis did not reveal any significant change on steady state activation (Va) or inactivation (Vh).

### EEG

Electroencephalography (EEG) is a non-invasive method to record overall brain activity from the skull surface in terms of general field potentials that can assess the relative power of EEG frequencies associated with different brain states or disorders such as epilepsy. A standard and rapid form of assessment is to calculate a power spectral density function to identify the relative contribution of EEG frequencies spanning from low frequencies (ie delta) found in synchronous states like sleeping, to the highest form (gamma) that characterizes high frequency desynchronized states. It has become increasingly recognized that FXS patients and Fmr1 KO mice exhibit higher than normal power in the gamma frequency (>30 Hz) that correlates with the sensory processing difficulties^{7,44-51}. Other studies have reported decreased alpha and increased theta power (Lovelace et al., 2018; van der Molen et al., 2014; Wang et al., 2017).

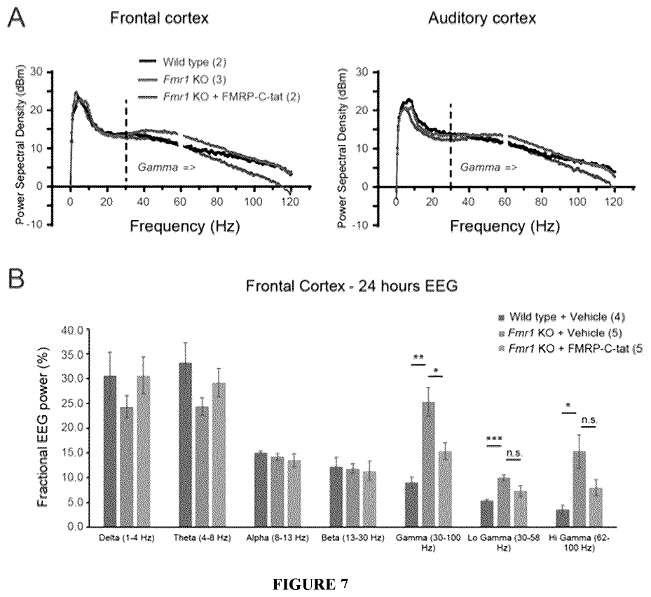

To test the effects of FMRP-C-tat on overall brain activity we prepared WT and Fmr1 KO mice for EEG recordings by mounting skull surface electrodes by stereotaxic surgery that were connected to a mounted head cap. After 1 week recovery from surgery animals were connected through the head cap to a Pinnacle EEG recording system and allowed to roam unimpeded in an open field while recording EEG activity for up to 3 days. EEG recordings of 5-10 min were collected for up to 3 days following tail vein injections. Power spectral density of EEG recorded from frontal cortex and auditory cortex revealed a normal range of EEG frequencies for WT mice injected with vehicle alone, but that from female Fmr1 KO mice at P60-P90 injected with vehicle alone exhibited an elevation of EEG power in the gamma frequency bands (30-100 Hz) (FIG. 7A, B). Tail vein injection of FMRP-C-tat(0.8 mg/kg) reduced the elevated levels of gamma frequencies in the EEG of Fmr1 KO even below that of WT mice within 3 hr of injection of FMRP-C-tat(FIG. 7A, B). EEG recordings from male Fmr1 KO mice showed similar rescue effects by FMRP-C-tat(0.8 mg/kg) at 24 hr post tail vein injection by reducing the gamma power in the EEG of the frontal cortex (FIG. 7B).

These data are important in revealing the ability for FMRP-C-tat to cross the BBB to restore overall brain circuit output in terms of EEG activity in the frontal cortex within 3 hr time that persists for at least 24 hr.

Summary: The preceding tests reveal that specific disorders inherent to Fragile X Syndrome that are replicated in the Fmr1 KO mouse model at the molecular, cellular and circuit function levels can be reduced or restored to near WT levels by reintroducing a 188 a.a. segment of the FMRP C-terminal region (aa 444-632). FMRP-C-tat introduced by tail vein injection in Fmr1 KO mice thus rapidly crosses the BBB to distribute as detectable immunolabel in cerebellar and cortical neurons at 24 hr, modified Kv4.2 potassium channel properties, and reduced enhanced levels of EEG gamma frequencies at 3 and 24 hr post injection. Direct application of FMRP-C-tat to the culture medium of HEK cells further indicated rapid uptake within that was retained at detectable levels for at least 24 hr. In each case the intensity of label for the associated myc or HA tags was directly dependent on concentration. Together the data support the potential for FMRP-C-tat to act as a potential therapeutic to reduce the cellular disorders inherent to Fragile X Syndrome.

### REFERENCES

1. Darnell, J. C. et al. FMRP stalls ribosomal translocation on mRNAs linked to synaptic function and autism. Cell 146, 247-261 (2011).
2. Belagodu, A. P., Johnson, A. M. & Galvez, R. Characterization of ultrasonic vocalizations of Fragile X mice. Behav. Brain Res. 310, 76-83 (2016).
3. Lai, J. K. et al. Temporal and spectral differences in the ultrasonic vocalizations of fragile X knock out mice during postnatal development. Behav. Brain Res. 259, 119-130 (2014).
4. Mineur, Y. S., Huynh, L. X. & Crusio, W. E. Social behavior deficits in the Fmr1 mutant mouse. Behav. Brain Res. 168, 172-175 (2006).
5. Ventura, R., Pascucci, T., Catania, M. V., Musumeci, S. A. & Puglisi-Allegra, S. Object recognition impairment in Fmr1 knockout mice is reversed by amphetamine: involvement of dopamine in the medial prefrontal cortex. Behav. Pharmacol. 15, 433-442 (2004).
6. Darnell, J. C. et al. Fragile X mental retardation protein targets G quartet mRNAs important for neuronal function. Cell 107, 489-499 (2001).
7. Wen, T. H., Lovelace, J. W., Ethell, I. M., Binder, D. K. & Razak, K. A. Developmental Changes in EEG Phenotypes in a Mouse Model of Fragile X Syndrome. Neuroscience 398, 126-143 (2019).
8. Zhan, X. et al. FMRP(1-297)-tat restores ion channel and synaptic function in a model of Fragile X syndrome. Nat. Commun. 11, 2755 (2020).
9. Bear, M. F., Huber, K. M. & Warren, S. T. The mGluR theory of fragile X mental retardation. Trends Neurosci. 27, 370-377 (2004).
10. Contractor, A., Klyachko, V. A. & Portera-Cailliau, C. Altered Neuronal and Circuit Excitability in Fragile X Syndrome. Neuron 87, 699-715 (2015).
11. Siegel, J. J. et al. Prefrontal Cortex Dysfunction in Fragile X Mice Depends on the Continued Absence of Fragile X Mental Retardation Protein in the Adult Brain. J. Neurosci. 37, 7305-7317 (2017).
12. Kalmbach, B. E., Johnston, D. & Brager, D. H. Cell-Type Specific Channelopathies in the Prefrontal Cortex of the fmr1-/y Mouse Model of Fragile X Syndrome. eNeuro 2, (2015).
13. Yau, S. Y., Chiu, C., Vetrici, M. & Christie, B. R. Chronic minocycline treatment improves social recognition memory in adult male Fmr1 knockout mice. Behav. Brain Res. 312, 77-83 (2016).
14. Martin, H. G. S., Lassalle, O., Brown, J. T. & Manzoni, O. J. Age-Dependent Long-Term Potentiation Deficits in the Prefrontal Cortex of the Fmr1 Knockout Mouse Model of Fragile X Syndrome. Cereb. Cortex 26, 2084-2092 (2016).
15. Kramvis, I., Mansvelder, H. D., Loos, M. & Meredith, R. Hyperactivity, perseveration and increased responding during attentional rule acquisition in the Fragile X mouse model. Front. Behav. Neurosci. 7, 172 (2013).
16. Bernardet, M. & Crusio, W. E. Fmr1 KO mice as a possible model of autistic features. ScientificWorldJournal 6, 1164-1176 (2006).
17. Graef, J. D. et al. Partial FMRP expression is sufficient to normalize neuronal hyperactivity in Fragile X neurons. Eur. J. Neurosci. (2019) doi: 10.1111/ejn.14660.
18. Reis, S. A., Willemsen, R., van Unen, L., Hoogeveen, A. T. & Oostra, B. A. Prospects of TAT-mediated protein therapy for fragile X syndrome. J. Mol. Histol. 35, 389-395 (2004).
19. Bowling, H. et al. Altered steady state and activity-dependent de novo protein expression in fragile X syndrome. Nat. Commun. 10, 1710 (2019).
20. Golden, C. E. M. et al. Deletion of the KH1 Domain of Fmr1 Leads to Transcriptional Alterations and Attentional Deficits in Rats. Cereb. Cortex 29, 2228-2244 (2019).
21. Brown, V. et al. Purified recombinant Fmrp exhibits selective RNA binding as an intrinsic property of the fragile X mental retardation protein. J. Biol. Chem. 273, 15521-15527 (1998).
22. Laggerbauer, B., Ostareck, D., Keidel, E. M., Ostareck-Lederer, A. & Fischer, U. Evidence that fragile X mental retardation protein is a negative regulator of translation. Hum. Mol. Genet. 10, 329-338 (2001).
23. Ramos, A. et al. The structure of the N-terminal domain of the fragile X mental retardation protein: a platform for protein-protein interaction. Structure 14, 21-31 (2006).
24. Siomi, M. C., Zhang, Y., Siomi, H. & Dreyfuss, G. Specific sequences in the fragile X syndrome protein FMR1 and the FXR proteins mediate their binding to 60S ribosomal subunits and the interactions among them. Mol. Cell. Biol. 16, 3825-3832 (1996).
25. Bartley, C. M. et al. Mammalian FMRP S499 Is Phosphorylated by CK2 and Promotes Secondary Phosphorylation of FMRP. eNeuro 3, (2016).
26. Bartley, C. M., O'Keefe, R. A. & Bordey, A. FMRP S499 is phosphorylated independent of mTORC1-S6K1 activity. PLOS One 9, e96956 (2014).
27. Spencer, K. B., Mulholland, P. J. & Chandler, L. J. FMRP Mediates Chronic Ethanol-Induced Changes in NMDA, Kv4.2, and KChIP3 Expression in the Hippocampus. Alcohol. Clin. Exp. Res. 40, 1251-1261 (2016).
28. Siomi, H., Siomi, M. C., Nussbaum, R. L. & Dreyfuss, G. The protein product of the fragile X gene, FMR1, has characteristics of an RNA-binding protein. Cell 74, 291-298 (1993).
29. Ferron, L., Nieto-Rostro, M., Cassidy, J. S. & Dolphin, A. C. Fragile X mental retardation protein controls synaptic vesicle exocytosis by modulating N-type calcium channel density. Nat. Commun. 5, 3628 (2014).
30. Eberhart, D. E., Malter, H. E., Feng, Y. & Warren, S. T. The fragile X mental retardation protein is a ribonucleoprotein containing both nuclear localization and nuclear export signals. Hum. Mol. Genet. 5, 1083-1091 (1996).
31. Adinolfi, S. et al. Dissecting FMR1, the protein responsible for fragile X syndrome, in its structural and functional domains. RNA 5, 1248-1258 (1999).
32. Fernandez, E., Rajan, N. & Bagni, C. The FMRP regulon: from targets to disease convergence. Front. Neurosci. 7, 191 (2013).
33. Di Marino, D., Achsel, T., Lacoux, C., Falconi, M. & Bagni, C. Molecular dynamics simulations show how the FMRP Ile304Asn mutation destabilizes the KH2 domain structure and affects its function. J. Biomol. Struct. Dyn. 32, 337-350 (2014).
34. Adinolfi, S. et al. The N-terminus of the fragile X mental retardation protein contains a novel domain involved in dimerization and RNA binding. Biochemistry 42, 10437-10444 (2003).
35. Ashley, C. T., Jr, Wilkinson, K. D., Reines, D. & Warren, S. T. FMR1 protein: conserved RNP family domains and selective RNA binding. Science 262, 563-566 (1993).
36. Menon, L. & Mihailescu, M.-R. Interactions of the G quartet forming semaphorin 3F RNA with the RGG box domain of the fragile X protein family. Nucleic Acids Res. 35, 5379-5392 (2007).
37. Fu, X. et al. Alternatively spliced products lacking exon 12 dominate the expression of fragile X mental retardation 1 gene in human tissues. Mol. Med. Rep. 12, 1957-1962 (2015).
38. Pretto, D. I. et al. Differential increases of specific FMR1 mRNA isoforms in premutation carriers. J. Med. Genet. 52, 42-52 (2015).
39. Hooper, A. W. M. et al. Gene therapy using an ortholog of human fragile X mental retardation protein partially rescues behavioral abnormalities and EEG activity. Mol Ther Methods Clin Dev 22, 196-209 (2021).
40. De Boulle, K. et al. A point mutation in the FMR-1 gene associated with fragile X mental retardation. Nat. Genet. 3, 31-35 (1993).
41. Musco, G. et al. Three-dimensional structure and stability of the KH domain: molecular insights into the fragile X syndrome. Cell 85, 237-245 (1996).
42. Tamanini, F. et al. Different targets for the fragile X-related proteins revealed by their distinct nuclear localizations. Hum. Mol. Genet. 8, 863-869 (1999).
43. Rizwan, A. P., Zhan, X., Zamponi, G. W. & Turner, R. W. Long-term potentiation at the mossy fiber-granule cell relay invokes postsynaptic second-messenger regulation of Kv4 channels. J. Neurosci. 36, 11196-11207 (2016).
44. Lovelace, J. W., Ethell, I. M., Binder, D. K. & Razak, K. A. Translation-relevant EEG phenotypes in a mouse model of Fragile X Syndrome. Neurobiol. Dis. 115, 39-48 (2018).
45. Ethridge, L. E. et al. Neural synchronization deficits linked to cortical hyper-excitability and auditory hypersensitivity in fragile X syndrome. Mol. Autism 8, 22 (2017).
46. Ethridge, L. E. et al. Reduced habituation of auditory evoked potentials indicate cortical hyper-excitability in Fragile X Syndrome. Transl. Psychiatry 6, e787 (2016).
47. Ethridge, L. E. et al. Auditory EEG Biomarkers in Fragile X Syndrome: Clinical Relevance. Front. Integr. Neurosci. 13, 60 (2019).
48. Wang, J. et al. A resting EEG study of neocortical hyperexcitability and altered functional connectivity in fragile X syndrome. J. Neurodev. Disord. 9, 11 (2017).
49. Sinclair, D., Oranje, B., Razak, K. A., Siegel, S. J. & Schmid, S. Sensory processing in autism spectrum disorders and Fragile X syndrome-From the clinic to animal models. Neurosci. Biobehav. Rev. 76, 235-253 (2017).
50. Molen, M. J. W. V. der, Van der Molen, M. J. W. & Van der Molen, M. W. Reduced alpha and exaggerated theta power during the resting-state EEG in fragile X syndrome. Biological Psychology vol. 92 216-219 Preprint at https://doi.org/10.1016/j.biopsycho.2012.11.013 (2013).
51. van der Molen, M. J. W., Stam, C. J. & van der Molen, M. W. Resting-state EEG oscillatory dynamics in fragile X syndrome: abnormal functional connectivity and brain network organization. PLOS One 9, e88451 (2014).
52. Xie, W., Dolzhanskaya, N., LaFauci, G., Dobkin, C. & Denman, R. B. Tissue and developmental regulation of fragile X mental retardation 1 exon 12 and 15 isoforms. Neurobiol. Dis. 35, 52-62 (2009).
53. Myrick, L. K., Hashimoto, H., Cheng, X. & Warren, S. T. FMRP N-terminal domain. Preprint at https://doi.org/10.2210/pdb4qvz/pdb (2014).

### Example 2

### EEG

Electroencephalography (EEG) is a non-invasive method to record overall brain activity from the skull surface in terms of general field potentials that can assess the relative power of EEG frequencies associated with different brain states or disorders such as epilepsy. A standard and rapid form of assessment is to calculate a Power Spectral Density (PSD) function to identify the relative power of EEG frequencies spanning from low frequencies (ie delta) found in synchronous states like sleeping, to the highest form (gamma) that characterizes high frequency desynchronized states. The most common report in cortical EEG of FXS subjects and Fmr1 KO rodent models is a higher power in gamma frequencies¹⁻⁵. This is important in that higher gamma power correlates with hyperexcitability, difficulties in sensory processing, and reductions in cognition^{2,4-8}. Other studies have reported altered levels of theta that reflect a disruption in theta-gamma coupling necessary for sensory processing and cognitive discrimination in both FXS subjects and the Fmr1 mouse^{4,9-11}. Further differences in EEG have been noted between males and females^{1,12-15}, and in the properties of EEG through development¹⁻⁵. Indeed, the degree of correspondence between EEG patterns in FXS subjects and rodent models has led EEG to be recognized as a biomarker for FXS^{6,16}.

To test the effects of FMRP-C-tat on overall brain activity we prepared male P60-90 WT and Fmr1 KO mice for EEG recordings by mounting skull surface electrodes through stereotaxic surgery that were connected to a head cap according to Standard Operating Procedures approved by the Animal Care Committee. Skull electrodes were positioned as previously described¹⁷ over the region of frontal cortex or auditory cortex at positions relative to bregma (anterior/posterior, medial/lateral in mm): reference electrode (−3.5, −3.5), ground (+2.0, +1.0), frontal cortex (+2.0, −1.0), auditory cortex (−1.6, +3.5). After sealing the connections and securing the head cap with dental cement the animals were allowed to recover for 7 days in a halfway house in the Animal Care Center. Mice were then connected under light isoflurane anesthesia to a wired Medusa EEG recording system and allowed to roam unimpeded in an open field while obtaining continuous differential EEG recordings for up to 3 days with simultaneous video recordings by overhead camera. In posthoc analysis a direct review of video recordings was used to confirm that mice were physically active or resting and awake to extract 5-10 min segments of EEG at intervals of 2, 6, 12, 24, 36, 48, 60, and 72 hr. EEG data shown here was drawn from male WT and Fmr1 KO mice, and with tail vein injections of FMRP-C-tat(0.8 mg/kg; isoform 1) or FMRP-N-tat(1.0 mg/kg; isoform 1)

### FMRP-C-Tat and FMRP-N-Tat Injections Differentially Affect PSD Measures of Resting EEG in Frontal and Auditory Cortex

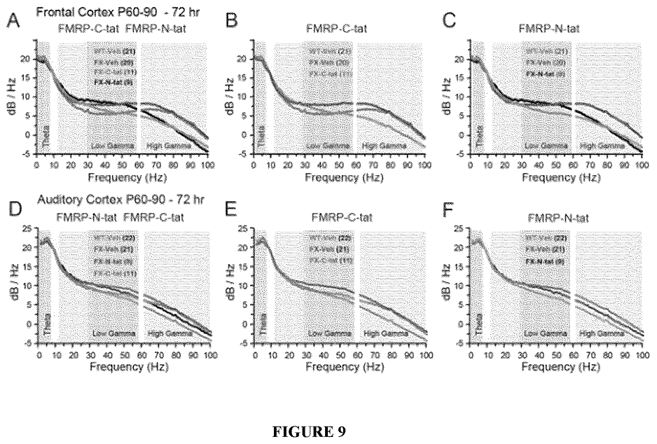

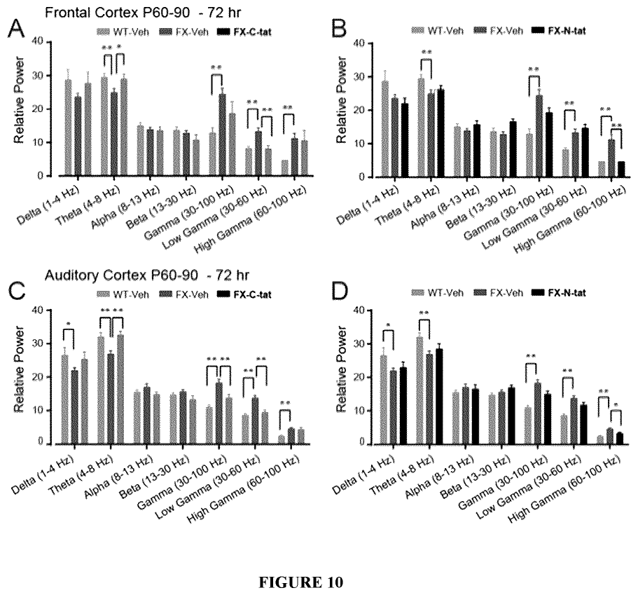

PSD plots of resting EEG recorded from the frontal cortex and auditory cortex revealed a typical range of EEG frequencies for WT mice injected with vehicle alone, in which a high power of low frequency signals dissipates to lower values at higher frequencies. Comparisons were made between animals tail-vein injected with either vehicle, FMRP-C-tat, or FMRP-N-tat for a point of comparison with a previously developed FMRP-tat peptide. Superimposition of PSD plots for Fmr1 KO mice injected with vehicle alone exhibited an elevated EEG power in the low gamma (30-60 Hz) and high gamma (60-100 Hz) frequency bands in both the frontal and auditory EEG recordings (FIG. 9A, D). In the frontal cortex injections of FMRP-C-tat reduced the elevated levels of low gamma frequencies in Fmr1 KO mice (FIG. 9A, B). Note that additional effects were detected in the theta frequency band upon calculating mean values (see FIG. 10). By comparison, injections of FMRP-N-tat primarily reduced high gamma frequencies in Fmr1 KO mice (FIG. 9C).

Similar recordings of EEG were collected over auditory regions of cortex after injections of vehicle, FMRP-C-tat or FMRP-N-tat and mean PSD plots for WT and Fmr1 KO mice calculated 72 hr after injections (FIG. 9D-F). Visual inspection of the superimposed PSD profiles for each record in the auditory cortex reveals that FMRP-C-tat injections also primarily rescued low gamma frequencies compared to WT animals injected with vehicle alone, but not higher gamma frequencies (FIG. 9EC). By comparison, injection of FMRP-N-tat restored the PSD profile for Fmr1 KO mice to that of WT mice in the range of high gamma frequencies but not low gamma frequencies (FIG. 9F).

### FMRP-C-Tat and FMRP-N-Tat Injections Exert Differential Rescue of Resting EEG Frequencies in Frontal and Auditory Cortex

Mean bar plots for different ranges of EEG frequencies based on the PSD calculations in FIG. 9 are shown in FIG. 10. The mean resting values of EEG are separated into frequencies corresponding to Delta (1-4 Hz), Theta (4-8 Hz), Alpha (8-13 Hz), Beta (13-30 Hz), and Gamma (30-100 Hz) frequencies, with additional consideration of low Gamma (30-60 Hz) and high Gamma (60-100 Hz) frequency ranges. These data emphasize how the effects exerted by these two peptides are not non-specific, but are restricted to specific EEG frequency ranges. Resting EEG in both the frontal and auditory cortex of Fmr1 KO mice exhibited a significantly lower level of theta frequencies and higher gamma frequencies, as well as lower delta frequencies in the auditory cortex compared to WT vehicle injected mice (FIG. 10A-D). As initially indicated by PSD plots, injection of FMRP-C-tat(FIG. 10A, C) selectively rescued theta and low gamma frequencies but not high gamma frequencies in both frontal and auditory cortex. By comparison, injection of FMRP-N-tat(FIG. 10B, D) selectively rescued high gamma frequencies in both frontal and auditory cortex, but not theta or low gamma frequencies.

### FMRP-C-Tat Injections Exert Long Term Rescue of Select EEG Frequencies in the Frontal Cortex

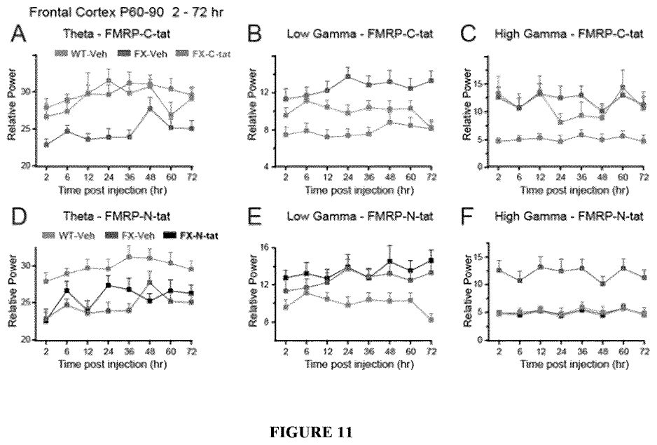

We calculated mean scatter plots of the relative power for theta, low gamma and high gamma frequencies over 3 days recording time following single injections of FMRP-C-tat(FIG. 11A-C) or FMRP-N-tat(FIG. 11D-F). FMRP-C-tat injection invoked rescue within 2 hr and throughout the following 72 hr for theta and low gamma frequencies (FIG. 11A, B) but not for high gamma frequencies (FIG. 11C). At a dose of 500 nM the effects of FMRP-C-tat injection exceeded the baseline resting values for low gamma frequencies of WT vehicle injected mice throughout most of 72 hr (FIG. 1B). In contrast, injection of FMRP-N-tat promoted full rescue of high gamma frequencies within 2-72 hr, but not for either theta or low gamma frequencies (FIG. 11D-F).

FIG. 11. FMRP-C-tat and FMRP-N-tat exert long-term effects on specific EEG frequencies in the frontal cortex of Fmr1 KO mice. Shown are mean scatter plots of the indicated EEG frequency ranges derived from PSD plots recorded in the frontal cortex at the indicated times after tail vein injection of vehicle in WT or Fmr1 KO (FX) mice (A-F), or FMRP-C-tat(FX-C-tat, 0.8 mg/kg) (A-C) or FMRP-N-tat(FX-N-tat, 1.0 mg/kg) (D-F) in Fmr1 KO mice. All recordings are from male P60-90 mice and drawn from data sets shown in FIG. 9. A-C, FMRP-C-tat injections rescue theta (A) and low gamma (B) frequencies within 2 hr injection, but not high gamma frequencies (C). Both theta and low gamma frequency rescue by FMRP-C-tat is intact 3 days following a single injection. D-F, FMRP-N-tat injection does not rescue either theta (D) or low gamma (E) frequencies, but achieves full rescue of high gamma frequencies (F) within 2 hr that persists throughout a 72 hr recording period. Average values are mean±SEM, with the number of samples for each group shown in FIG. 9.

### FMRP-C-Tat Injections Exert Long Term Rescue of Select EEG Frequencies in Auditory Cortex

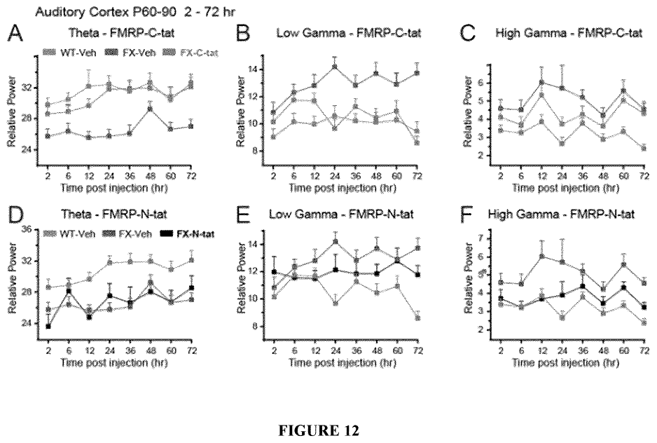

Mean scatter plots of the calculated relative power for theta, low gamma and high gamma frequencies in auditory cortex over 3 days recording time following single injections of FMRP-C-tat (FIG. 12A-C) or FMRP-N-tat (FIG. 12D-F). Similar to the frontal cortex (FIG. 11), FMRP-C-tat injection invoked rescue of EEG frequencies in auditory cortex within 2 hr and throughout the following 72 hr for both theta and low gamma frequencies (FIG. 12A, B) but less effectively for high gamma frequencies compared to the frontal cortex (cf. FIGS. 11C, 12C). In contrast, injection of FMRP-N-tat promoted rescue of high gamma frequencies within 2-72 hr, but not for either theta or low gamma frequencies (FIG. 12D-F).

FIG. 12. FMRP-C-tat and FMRP-N-tat exert long-term effects on specific EEG frequencies in the auditory cortex of Fmr1 KO mice. Shown are mean scatter plots of the indicated EEG frequency ranges derived from PSD plots recorded in the auditory cortex at the indicated times after tail vein injection of vehicle in WT or Fmr1 KO (FX) mice (A-F), or FMRP-C-tat (FX-C-tat, 0.8 mg/kg) (A-C) or FMRP-N-tat (FX-N-tat, 1.0 mg/kg) (D-F) in Fmr1 KO mice. All recordings are from male P60-90 mice and drawn from data sets shown in FIG. 9. A-C, FMRP-C-tat injections rescue theta (A) and low gamma (B) frequencies within 2 hr injection, but with less efficacy for high gamma frequencies (C). Both theta and low gamma frequency rescue by FMRP-C-tat is intact 3 days following a single injection. D-F, FMRP-N-tat injection does not rescue theta (D) frequency, has variable rescue of low gamma (E) frequencies, but a more convincing rescue of high gamma frequencies (F) within 2 hr and throughout a 72 hr recording period. Average values are mean±SEM, with the number of samples for each group shown in FIG. 7.

### FMRP-C-Tat and FMRP-N-Tat Exert Complementary Rescue of Cortical EEG Frequencies

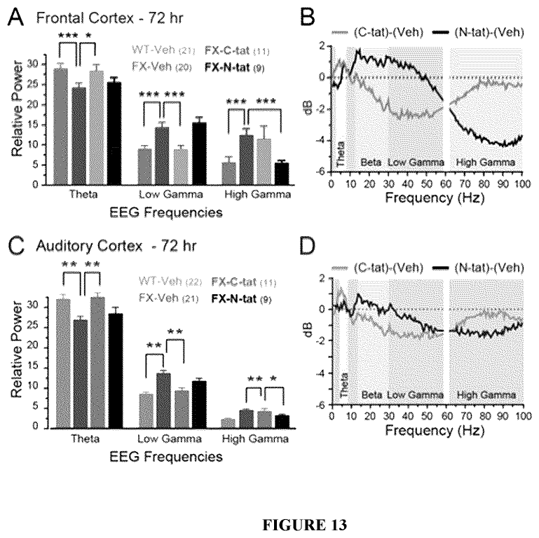

A comparison of the effects of injecting FMRP-C-tat or FMRP-N-tat on EEG is compiled in FIG. 13 by focusing on the effects of these peptides in the range of theta, low gamma, and high gamma frequencies 72 hr after injections. Mean bar plots of relative PSD power in the frontal cortex emphasizes how FMRP-C-tat provides a convincing rescue of both theta and low gamma frequencies but not high gamma frequencies (FIG. 13A). Conversely, the rescue effects of FMRP-N-tat in the frontal cortex are preferentially for the higher range of gamma frequencies (FIG. 13A). Particularly revealing is a calculation of the relative degree of rescue by either peptide over the full range of PSD power calculations, as determined by subtracting the mean PSD for vehicle injected Fmr1 KO mice from that of FMRP-C-tat or FMRP-N-tat recordings at 72 hr. Here it becomes clear that FMRP-C-tat rescues theta EEG, some initial rescue of beta frequencies that becomes maximal during low gamma frequencies but dissipates into the high gamma frequency range (FIG. 13B). The curve for FMRP-N-tat is almost the mirror image of that for FMRP-C-tat in showing some rescue in the beta frequency range, less through low gamma frequencies, and maximal effect for the high gamma frequencies (FIG. 13B).

A comparison of the effects of these peptides in the auditory cortex returned similar results, although with notable differences. The mean bar plots for FMRP-C-tat in the auditory cortex were essentially equivalent to that of the frontal cortex with clear effects in rescuing theta frequencies. While FMRP-N-tat exerted moderate rescue of low gamma frequencies there was less pronounced influence on high gamma frequencies (cf. FIG. 13A, C). Calculating the difference between drug- and vehicle-treated Fmr1 KO PSD plots highlighted these differences (FIG. 13D). In general, both FMRP-C-tat and FMRP-N-tat injections evoked a lower relative magnitude of rescue in the auditory cortex compared to the frontal cortex. Yet the pronounced effect of FMRP-C-tat in rescuing theta frequencies was apparent in the auditory cortex, with a similar distribution of rescue over the range of beta to high gamma frequencies (FIG. 13D). The influence of FMRP-N-tat in the auditory cortex was overall more muted and with a shifted profile compared to the frontal cortex that revealed some effects on both low and high gamma frequencies.

FIG. 13. FMRP-C-tat and FMRP-N-tat exert complementary rescue of select cortical EEG frequencies in Fmr1 KO mice for at least 3 days. A, C, Summary mean bar plots of the indicated EEG frequency ranges comparing the rescue effects of FMRP-tat peptides in the frontal cortex (A) and auditory cortex (C). Data represent EEG recorded 72 hr after tail vein injection of vehicle in WT or Fmr1 KO (FX) mice, or FMRP-C-tat(FX-C-tat, 0.8 mg/kg) or FMRP-N-tat(FX-N-tat, 1.0 mg/kg) in Fmr1 KO mice. FMRP-C-tat acts to rescue theta frequencies and low gamma frequencies in both frontal (A) and auditory cortex (C) but not high gamma frequencies. In contrast, FMRP-N-tat injection does not rescue theta or low gamma frequencies in either frontal or auditory cortex, with actions focused instead on high gamma frequencies primarily in frontal cortex. B, D, Superimposed mean PSD plots of the frequencies of EEG rescued by FMRP-C-tat or FMRP-N-tat injections in the frontal cortex (B) or auditory cortex (D). PSD representations were calculated through subtraction of mean PSD values for drug treated vs vehicle injected Fmr1 KO mice and presented on the same scale. A complementarity of rescue effects by FMRP-C-tat and FMRP-N-tat in the frontal cortex is apparent in almost mirrored representations of EEG frequencies rescued by 72 hr after injections (B). D, FMRP-C-tat and FMRP-N-tat have a similar pattern of effects on EEG frequencies in the auditory cortex as in the frontal cortex, but of lower relative magnitude exerted over a broader range of frequencies than in frontal cortex.

Summary: The preceding tests reveal that specific disorders inherent to Fragile X Syndrome that are replicated in the Fmr1 KO mouse model at the molecular, cellular and circuit function levels can be reduced or restored to near WT levels by reintroducing a 188 a.a. segment of the FMRP C-terminal region (aa 444-630, isoform 1). FMRP-C-tat introduced by tail vein injection in Fmr1 KO mice thus rapidly crosses the BBB to distribute as detectable immunolabel in cerebellar and cortical neurons at 24 hr, modified Kv4.2 potassium channel properties. Direct application of FMRP-C-tat to the culture medium of HEK cells further indicated rapid uptake within that was retained at detectable levels for at least 24 hr. In each case the intensity of label for the associated myc or HA tags was directly dependent on concentration. The effects of FMRP-C-tat on EEG recordings are particularly interesting in that EEG is recognized as a powerful biomarker for FXS, with the ability to monitor therapeutic interventions in either FXS subjects or the Fmr1 KO mouse model. FMRP-C-tat proved to restore multiple aspects of altered levels of EEG frequencies in the Fmr1 KO mouse within 2 hr. Moreover, the rescue of EEG in almost all cases persisted without return to KO levels for at least 3 days. FMRP-C-tat actions were directed primarily to a wide range of lower EEG frequency bands (theta, beta, low gamma) compared to high gamma frequencies, with more pronounced effects in the frontal cortex than auditory cortex. A comparison to the actions of FMRP-N-tat further indicated an almost complementary effect to that of FMRP-N-tat that primarily reduced altered levels of high gamma frequencies compared to lower frequencies. Comparison of the relative efficacy of these constructs between the frontal and auditory cortex revealed a similar overall influence of both constructs but with different relative magnitudes. The greater effect of both FMRP-C-tat and FMRP-N-tat in the frontal cortex is promising in their potential to alleviate the prominent symptoms of lower function in cognition and social interactions in FXS subjects. Effects on the auditory cortex by these constructs would be predicted to alleviate the hypersensitivity of FXS subjects to sensory stimuli. Together the data support the potential for FMRP-C-tat to act as a potential therapeutic to reduce the cellular disorders inherent to Fragile X Syndrome, and with complementary effects to that of FMRP-N-tat, findings that could be used to advantage through joint application of these agents.

### REFERENCES

1. Wong, H. et al. Sexually dimorphic patterns in electroencephalography power spectrum and autism-related behaviors in a rat model of fragile X syndrome. Neurobiol. Dis. 146, 105118 (2020).
2. Wen, T. H., Lovelace, J. W., Ethell, I. M., Binder, D. K. & Razak, K. A. Developmental Changes in EEG Phenotypes in a Mouse Model of Fragile X Syndrome. Neuroscience 398, 126-143 (2019).
3. Kozono, N., Okamura, A., Honda, S., Matsumoto, M. & Mihara, T. Gamma power abnormalities in a Fmr1-targeted transgenic rat model of fragile X syndrome. Sci. Rep. 10, 18799 (2020).
4. Wang, J. et al. A resting EEG study of neocortical hyperexcitability and altered functional connectivity in fragile X syndrome. J. Neurodev. Disord. 9, 11 (2017).
5. Gonçalves, J. T., Anstey, J. E., Golshani, P. & Portera-Cailliau, C. Circuit level defects in the developing neocortex of Fragile X mice. Nat. Neurosci. 16, 903-909 (2013).
6. Lovelace, J. W., Ethell, I. M., Binder, D. K. & Razak, K. A. Translation-relevant EEG phenotypes in a mouse model of Fragile X Syndrome. Neurobiol. Dis. 115, 39-48 (2018).
7 Sinclair, D., Oranje, B., Razak, K. A., Siegel, S. J. & Schmid, S. Sensory processing in autism spectrum disorders and Fragile X syndrome-From the clinic to animal models. Neurosci. Biobehav. Rev. 76, 235-253 (2017).
8. Orekhova, E. V. et al. Sensory gating in young children with autism: relation to age, IQ, and EEG gamma oscillations. Neurosci. Lett. 434, 218-223 (2008).
9. Ethridge, L. E. et al. Neural synchronization deficits linked to cortical hyper-excitability and auditory hypersensitivity in fragile X syndrome. Mol. Autism 8, 22 (2017).
10. Hooper, S. R. et al. Executive functions in young males with fragile X syndrome in comparison to mental age-matched controls: baseline findings from a longitudinal study. Neuropsychology 22, 36-47 (2008).
11. Brennan, F. X., Albeck, D. S. & Paylor, R. Fmr1 knockout mice are impaired in a leverpress escape/avoidance task. Genes Brain Behav. 5, 467-471 (2006).
12. Goswami, S., Cavalier, S., Sridhar, V., Huber, K. M. & Gibson, J. R. Local cortical circuit correlates of altered EEG in the mouse model of Fragile X syndrome. Neurobiology of Disease vol. 124 563-572 Preprint at https://doi.org/10.1016/j.nbd.2019.01.002 (2019).
13. Hooper, A. W. M. et al. Gene therapy using an ortholog of human fragile X mental retardation protein partially rescues behavioral abnormalities and EEG activity. Mol Ther Methods Clin Dev 22, 196-209 (2021).
14. Sinclair, D. et al. GABA-B Agonist Baclofen Normalizes Auditory-Evoked Neural Oscillations and Behavioral Deficits in the Fmr1 Knockout Mouse Model of Fragile X Syndrome. eNeuro 4, (2017).
15. Smith, E. G. et al. Sex differences in resting EEG power in Fragile X Syndrome. J. Psychiatr. Res. 138, 89-95 (2021).
16. Kenny, A., Wright, D. & Stanfield, A. C. EEG as a translational biomarker and outcome measure in fragile X syndrome. Transl. Psychiatry 12, 34 (2022).
17. Murari, K., Abushaibah, A., Rho, J. M., Turner, R. W. & Cheng, N. A clinically relevant selective ERK-pathway inhibitor reverses core deficits in a mouse model of autism. EBioMedicine 91, 104565 (2023).

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A recombinant fusion polypeptide, comprising: a FMRP(444-632) polypeptide and a cell penetrating polypeptide, or fragment or variant thereof.

2. The recombinant fusion polypeptide of claim 1, where said cell penetrating polypeptide comprises a tat polypeptide.

3. The recombinant fusion polypeptide of claim 1 or 2, further comprising a linker positioned between said cell penetrating polypeptide and said FMRP(444-632) polypeptide.

4. The recombinant fusion polypeptide of claim 3, wherein the linker is GSGGG (SEQ ID NO:11).

5. The recombinant fusion polypeptide of any one of claims 2 to 4, where said tat polypeptide comprises YGRKKRRQRRR (SEQ ID NO: 4).

6. The recombinant fusion polypeptide of any one of claims 1 to 5, further comprising a HIS polypeptide.

7. The recombinant fusion polypeptide of claim 6, wherein said HIS polypeptide comprises HHHHHH (SEQ ID NO:12) or MGGSHHHHHHGMAS (SEQ ID NO: 5).

8. The recombinant fusion polypeptide of any one of claims 1 to 7, further comprising an immunogenic tag.

9. The recombinant fusion polypeptide of claim 8, wherein the immunogenic tag is a c-myc tag.

10. The recombinant fusion polypeptide of claim 9, wherein the c-myc tag is EQKLISEEDL (SED ID NO: 6).

11. The recombinant fusion polypeptide of any one of claims 1 to 10, wherein the FMRP(444-632) polypeptide is
(SEQ ID NO: 7)
MGASSRPPPNRTDKEKSYVTDDGQGMGRGSRPYRNRGHGRRGPGYTSGTNSEASNA
SETESDHRDELSDWSLAPTEEERESFLRRGDGRRRGGGGRGQGGRGRGGGFKGND
DHSRTDNRPRNPREAKGRTTDGSLQIRVDCNNERSVHTKTLQNTSSEGSRLRTGKDRN
QKKEKPDSVDGQQPLVNGVP.

12. The recombinant fusion polypeptide of any one of claims 1 to 11, wherein the sequence is
(SEQ ID NO: 3)
MGGSHHHHHHGMASEQKLISEEDLMGASSRPPPNRTDKEKSYVTDDGQGMGRGSRP
YRNRGHGRRGPGYTSGTNSEASNASETESDHRDELSDWSLAPTEEERESFLRRGDGR
RRGGGGRGQGGRGRGGGFKGNDDHSRTDNRPRNPREAKGRTTDGSLQNTSSEGSR
LRTGKDRNQKKEKPDSVDGQQPLVNGVPGSGGGYGRKKRRQRRR.

13. The recombinant fusion polypeptide of any one of claims 1 to 12, wherein said fusion polypeptide comprises a variant fusion polypeptide sequence that is at least 80% identical to said fusion polypeptide.

14. A polynucleotide molecule comprising or consisting of a sequence that encodes a recombinant fusion polypeptide according to any one of claims 1 to 13.

15. A vector comprising the polynucleotide molecule of claim 14.

16. A cell comprising the polynucleotide molecule of claim 14.

17. A cell comprising the vector of claim 15.

18. A pharmaceutical composition comprising a recombinant fusion polypeptide of any one of claims 1-13, a polynucleotide molecule of claim 14, or a vector of claim 15, and a pharmaceutically acceptable carrier.

19. A method of treatment of a subject having or suspected of having Fragile X Syndrome, comprising: administering a recombinant fusion polypeptide of any one of claims 1 to 13, a polynucleotide molecule of claim 14, a vector of claim 15, or a pharmaceutical composition of claim 18, to said subject.

20. The method of claim 19, further comprising administration of minocycline, metformin, and/or blockers of extracellular signal-regulated kinase (ERK).

21. The method of claim 19 or 20, wherein said subject is a human.

22. A recombinant fusion polypeptide, comprising: a FMRP(399-570) polypeptide and a cell penetrating polypeptide, or fragment or variant thereof.

23. The recombinant fusion polypeptide of claim 22, where said cell penetrating polypeptide comprises a tat polypeptide.

24. The recombinant fusion polypeptide of claim 22 or 23, further comprising a linker positioned between said cell penetrating polypeptide and said FMRP(399-570) polypeptide.

25. The recombinant fusion polypeptide of claim 24, wherein the linker is GSGGG (SEQ ID NO:11).

26. The recombinant fusion polypeptide of any one of claims 23 to 25, where said tat polypeptide comprises YGRKKRRQRRR (SEQ ID NO: 4).

27. The recombinant fusion polypeptide of any one of claims 22 to 26, further comprising a HIS polypeptide.

28. The recombinant fusion polypeptide of claim 27, wherein said HIS polypeptide comprises HHHHHH (SEQ ID NO: 12) or MGGSHHHHHHGMAS (SEQ ID NO: 5).

29. The recombinant fusion polypeptide of any one of claims 22 to 28, further comprising an immunogenic tag.

30. The recombinant fusion polypeptide of claim 29, wherein the immunogenic tag is a c-myc tag.

31. The recombinant fusion polypeptide of claim 30, wherein the c-myc tag is EQKLISEEDL (SEQ ID NO: 6).

32. The recombinant fusion polypeptide of any one of claims 22 to 31, wherein the FMRP(399-570) polypeptide is
(SEQ ID NO: 8)
MGASSRPPPNRTDKEKSYVTDDGQGMGRGSRPYRNRGHGRRGPGYTSGTNSEASNA
SETESDHRDELSDWSLAPTEEERESFLRRGDGRRRGGGGRGQGGRGRGGGFKGND
DHSRTDNRPRNPREAKGRTTDGSLQNTSSEGSRLRTGKDRNQKKEKPDSVDGQQPLV
NGVP.

33. The recombinant fusion polypeptide of any one of claims 22 to 32, wherein the sequence is
(SEQ ID NO: 3)
MGGSHHHHHHGMASEQKLISEEDLMGASSRPPPNRTDKEKSYVTDDGQGMGRGSRP
YRNRGHGRRGPGYTSGTNSEASNASETESDHRDELSDWSLAPTEEERESFLRRGDGR
RRGGGGRGQGGRGRGGGFKGNDDHSRTDNRPRNPREAKGRTTDGSLQNTSSEGSR
LRTGKDRNQKKEKPDSVDGQQPLVNGVPGSGGGYGRKKRRQRRR.

34. The recombinant fusion polypeptide of any one of claims 22 to 33, wherein said fusion polypeptide comprises a variant fusion polypeptide sequence that is at least 80% identical to said fusion polypeptide.

35. A polynucleotide molecule comprising or consisting of a sequence that encodes a recombinant fusion polypeptide according to any one of claims 22 to 34.

36. A vector comprising the polynucleotide molecule of claim 35.

37. A cell comprising the polynucleotide molecule of claim 35.

38. A cell comprising the vector of claim 36.

39. A pharmaceutical composition comprising a recombinant fusion polypeptide of any one of claims 22-34, a polynucleotide molecule of claim 35, or a vector of claim 36, and a pharmaceutically acceptable carrier.

40. A method of treatment of a subject having or suspected of having Fragile X Syndrome, comprising: administering a recombinant fusion polypeptide of any one of claims 22 to 34, a polynucleotide molecule of claim 35, a vector of claim 36, or a pharmaceutical composition of claim 39, to said subject.

41. The method of claim 40, further comprising administration of minocycline, metformin, and/or blockers of extracellular signal-regulated kinase (ERK).

42. The method of claim 40 or 41, wherein said subject is a human.

43. A recombinant fusion polypeptide, comprising: a FMRP(205-399) polypeptide and a cell penetrating polypeptide, or fragment or variant thereof.

44. The recombinant fusion polypeptide of claim 43, where said cell penetrating polypeptide comprises a tat polypeptide.

45. The recombinant fusion polypeptide of claim 43 or 44, further comprising a linker positioned between said cell penetrating polypeptide and said FMRP(205-399) polypeptide.

46. The recombinant fusion polypeptide of claim 45, wherein the linker is GSGGG (SEQ ID NO:11).

47. The recombinant fusion polypeptide of any one of claims 44 to 46, where said tat polypeptide comprises YGRKKRRQRRR (SEQ ID NO: 4).

48. The recombinant fusion polypeptide of any one of claims 43 to 47, further comprising a HIS polypeptide.

49. The recombinant fusion polypeptide of claim 48, wherein said HIS polypeptide comprises HHHHHH (SEQ ID NO: 12) or MGGSHHHHHHGMAS (SEQ ID NO: 5).

50. The recombinant fusion polypeptide of any one of claims 43 to 49, further comprising an immunogenic tag.

51. The recombinant fusion polypeptide of claim 50, wherein the immunogenic tag is a c-myc tag.

52. The recombinant fusion polypeptide of claim 51, wherein the c-myc tag is EQKLISEEDL (SEQ ID NO: 6).

53. The recombinant fusion polypeptide of any one of claims 43 to 52, wherein the FMRP(205-399) polypeptide is
(SEQ ID NOa: 1)
MSKQLESSRQLASRFHEQFIVREDLMGLAIGTHGANIQQARKVPGVTAIDLDEDTCTFHI
YGEDQDAVKKARSFLEFAEDVIQVPRNLVGKVIGKNGKLIQEIVDKSGVVRVRIEAENEK
NVPQEEVLVASSVVAGESQKPELKAWQGMVPFVFVGTKDSIANATVLLDYHLNYLKEVD
QLRLERLQIDEQLRQI.

54. The recombinant fusion polypeptide of any one of claims 43 to 53, wherein the sequence is
SEQ ID NO: 9)
MGGSHHHHHHGMASEQKLISEEDLMSKQLESSRQLASRFHEQFIVREDLMGLAIGTHG
ANIQQARKVPGVTAIDLDEDTCTFHIYGEDQDAVKKARSFLEFAEDVIQVPRNLVGKVIGK
NGKLIQEIVDKSGWVRVRIEAENEKNVPQEEVLVASSVVAGESQKPELKAWQGMVPFVF
VGTKDSIANATVLLDYHLNYLKEVDQLRLERLQIDEQLRQIGSGGGYGRKKRRQRRR.

55. The recombinant fusion polypeptide of any one of claims 43 to 54, wherein said fusion polypeptide comprises a variant fusion polypeptide sequence that is at least 80% identical to said fusion polypeptide, or fragments or variants thereof.

56. A polynucleotide molecule comprising or consisting of a sequence that encodes a recombinant fusion polypeptide according to any one of claims 43 to 55.

57. A vector comprising the polynucleotide molecule of claim 56.

58. A cell comprising the polynucleotide molecule of claim 56.

59. A cell comprising the vector of claim 57.

60. A pharmaceutical composition comprising a recombinant fusion polypeptide of any one of claims 43-55, a polynucleotide molecule of claim 56, or a vector of claim 57, and a pharmaceutically acceptable carrier.

61. A method of treatment of a subject having or suspected of having Fragile X Syndrome, comprising: administering a recombinant fusion polypeptide of any one of claims 43 to 55, a polynucleotide molecule of claim 56, a vector of claim 57, or a pharmaceutical composition of claim 60, to said subject.

62. The method of claim 61, further comprising administration of minocycline, metformin, and/or blockers of extracellular signal-regulated kinase (ERK).

63. The method of claim 61 or 62, wherein said subject is a human.

64. A method of treatment of a subject having or suspected of having Fragile X Syndrome, comprising:
administering a recombinant fusion polypeptide of any one of claims 1 to 13, a polynucleotide molecule of claim 14, a vector of claim 15, or a pharmaceutical composition of claim 18, to said subject and/or
administering a recombinant fusion polypeptide of any one of claims 22 to 34, a polynucleotide molecule of claim 35, a vector of claim 36, or a pharmaceutical composition of claim 39, to said subject, and/or
administering a recombinant fusion polypeptide of any one of claims 43 to 55, a polynucleotide molecule of claim 56, a vector of claim 57, or a pharmaceutical composition of claim 60, to said subject.

65. The method of claim 64, further comprising administration of minocycline, metformin, and/or blockers of extracellular signal-regulated kinase (ERK).

66. The method of claim 64 or 65, wherein said subject is a human.


SEQUENCE LISTING (12 sequences)

SEQ ID NO: 1 - protein, 195 aa, synthetic construct
MSKQLESSRQLASRFHEQFIVREDLMGLAIGTHGANIQQARKVPGVTAIDLDEDTCTFHIYGEDQDAVKKARSFLEFAEDVIQVPRNLVGKVIGKNGKLIQEIVDKSGVVRVRIEAENEKNVPQEEVLVASSVVAGESQKPELKAWQGMVPFVFVGTKDSIANATVLLDYHLNYLKEVDQLRLERLQIDEQLRQI

SEQ ID NO: 2 - protein, 229 aa, synthetic construct
MGGSHHHHHHGMASEQKLISEEDLMGASSRPPPNRTDKEKSYVTDDGQGMGRGSRPYRNRGHGRRGPGYTSGTNSEASNASETESDHRDELSDWSLAPTEEERESFLRRGDGRRRGGGGRGQGGRGRGGGFKGNDDHSRTDNRPRNPREAKGRTTDGSLQIRVDCNNERSVHTKTLQNTSSEGSRLRTGKDRNQKKEKPDSVDGQQPLVNGVPGSGGGYGRKKRRQRRR

SEQ ID NO: 3 - protein, 212 aa, synthetic construct
MGGSHHHHHHGMASEQKLISEEDLMGASSRPPPNRTDKEKSYVTDDGQGMGRGSRPYRNRGHGRRGPGYTSGTNSEASNASETESDHRDELSDWSLAPTEEERESFLRRGDGRRRGGGGRGQGGRGRGGGFKGNDDHSRTDNRPRNPREAKGRTTDGSLQNTSSEGSRLRTGKDRNQKKEKPDSVDGQQPLVNGVPGSGGGYGRKKRRQRRR

SEQ ID NO: 4 - protein, 11 aa, synthetic construct
YGRKKRRQRRR

SEQ ID NO: 5 - protein, 14 aa, synthetic construct
MGGSHHHHHHGMAS

SEQ ID NO: 6 - protein, 10 aa, synthetic construct
EQKLISEEDL

SEQ ID NO: 7 - protein, 189 aa, synthetic construct
MGASSRPPPNRTDKEKSYVTDDGQGMGRGSRPYRNRGHGRRGPGYTSGTNSEASNASETESDHRDELSDWSLAPTEEERESFLRRGDGRRRGGGGRGQGGRGRGGGFKGNDDHSRTDNRPRNPREAKGRTTDGSLQIRVDCNNERSVHTKTLQNTSSEGSRLRTGKDRNQKKEKPDSVDGQQPLVNGVP

SEQ ID NO: 8 - protein, 172 aa, synthetic construct
MGASSRPPPNRTDKEKSYVTDDGQGMGRGSRPYRNRGHGRRGPGYTSGTNSEASNASETESDHRDELSDWSLAPTEEERESFLRRGDGRRRGGGGRGQGGRGRGGGFKGNDDHSRTDNRPRNPREAKGRTTDGSLQNTSSEGSRLRTGKDRNQKKEKPDSVDGQQPLVNGVP

SEQ ID NO: 9 - protein, 235 aa, synthetic construct
MGGSHHHHHHGMASEQKLISEEDLMSKQLESSRQLASRFHEQFIVREDLMGLAIGTHGANIQQARKVPGVTAIDLDEDTCTFHIYGEDQDAVKKARSFLEFAEDVIQVPRNLVGKVIGKNGKLIQEIVDKSGVVRVRIEAENEKNVPQEEVLVASSVVAGESQKPELKAWQGMVPFVFVGTKDSIANATVLLDYHLNYLKEVDQLRLERLQIDEQLRQIGSGGGYGRKKRRQRRR

SEQ ID NO: 10 - protein, 9 aa, synthetic construct
YPYDVPDYA

SEQ ID NO: 11 - protein, 5 aa, synthetic construct
GSGGG

SEQ ID NO: 12 - protein, 6 aa, synthetic construct
HHHHHH